SEQUENCE LISTING (5 sequences)

SEQ ID NO: 1 - protein, 4 aa, Artificial Sequence
  protein ligase recognition sequence
  misc_feature (3)..(3): Xaa can be any naturally occurring amino acid
LPXT

SEQ ID NO: 2 - protein, 5 aa, Artificial Sequence
  protein ligase recognition sequence
  misc_feature (3)..(3): Xaa can be any naturally occurring amino acid
LPXTG

SEQ ID NO: 3 - protein, 5 aa, Artificial Sequence
  protein ligase recognition sequences
LPETG

SEQ ID NO: 4 - DNA, 22 bp, Artificial Sequence
  miRNA
acugauuucuuuugguguucag

SEQ ID NO: 5 - DNA, 20 bp, Artificial Sequence
  gRNA
ccucacaaguuagggucuca

(19) United States
(12) Patent Application Publication
(10) Pub. No.: US 2023/0121065 A1
(43) Pub. Date: Apr. 20, 2023

(54) METHOD OF DELIVERING NUCLEIC ACID TO IMMUNE CELLS USING RBCEV

(71) Applicants: National University of Singapore, Singapore (SG); Cornell University, Ithaca, NY (US)

(72) Inventors: Minh Thi Nguyet Le, Singapore (SG); Tien Luyen Vu, Singapore (SG); Andrew Grimson, Ithaca, NY (US); Brian Rudd, Ithaca, NY (US); Kristel Joy Yee Mon, Ithaca, NY (US); Tuan Thach Pham, Singapore (SG); Boya Peng, Singapore (SG)

(21) Appl. No.: 17/914,194

(22) PCT Filed: Mar. 25, 2021
(86) PCT No.: PCT/SG2021/050166

§ 371 Date: Sep. 23, 2022

Related U.S. Application Data
(60) Provisional application No. 63/000,468, filed on Mar. 26, 2020.

(51) Int. Cl. C12N 15/88; A61K 35/17; C12N 15/113; C12N 15/11

(52) U.S. Cl. CPC C12N 15/88; A61K 35/17; C12N 15/113; C12N 15/11; C12N 2310/113; C12N 2310/141; C12N 2320/32; C12N 2310/20

(57) ABSTRACT

The invention relates to the use of red blood cells extracellular vesicle (RBCEV) to deliver nucleic acids to immune cells (e.g. T cells), wherein the nucleic acid cargoes are loaded into the RBCEV in vitro or ex vivo. The invention also relates to the prophylactic and therapeutic use of said immune cells in diseases, such as cancer.

This application claims priority from U.S. 63/000,468 filed 26 Mar. 2020, the contents and elements of which are herein incorporated by reference for all purposes.

### FIELD OF THE INVENTION

The present invention relates to immune cells and particularly, although not exclusively, to methods of delivering agents, e.g. nucleic acid, to immune cells. The invention also relates to immune cells comprising said nucleic acid and the use of said immune cells in methods of medical treatment and prophylaxis.

### BACKGROUND

T lymphocytes are an essential component of the adaptive immune system which defend our body against infections and cancer. Therefore, it is important to understand the molecular mechanism of T cell development by altering the expression of genes involved in T cell activation and function. Manipulating and engineering T cells are also important for T cell therapies, including CAR T cells, adoptive T cell transfer, T cell receptor therapies, which are emerging as new treatments for cancer and other diseases.

Modification of T cells relies on viral and non-viral methods to deliver nucleic acids into these cells. Viral vectors often provide high transfection efficiency but it is lengthy, costly and labour-intensive to create high-quality virus. Moreover, lentiviral transduction often leads to DNA integration with a high risk of transformation in the host cells. Non-viral delivery methods including electroporation and nucleofection are faster and more economical. However, these treatments are harsh for the cells leading to high mortality rates. Other delivery methods such as adeno-associated virus transduction and lipofection are not effective for T cells. Therefore, there is a high demand for an effective and viable method for T cell transfection.

Extracellular vesicles (EVs) are nanosized cell-derived particles enclosed by a phospholipid bilayer membrane. They are secreted by all living cells and are divided into different subtypes including exosomes and microvesicles. Exosomes are generated from the inward budding of the endosomal membrane, forming intraluminal vesicles in multivesicular bodies that would eventually fuse with the plasma membrane and release exosomes into the extracellular space. Microvesicles are formed by directly budding off from the plasma membrane. Typically, exosomes are 30-100 nm in diameter, whereas microvesicles are larger than 100 nm.

EVs are comprised of different lipids, proteins (surface and intraluminal), and nucleic acids. Naturally, EVs act as a means for intercellular communication by interacting with recipient cells at the surface through receptor-ligand binding or intracellularly via endocytosis. Because of their natural ability to transport large macromolecules across the cell membrane, EVs have been proposed as drug delivery vehicles for the transport of small molecules, proteins and nucleic acids that include short RNAs like antisense oligonucleotides (ASOs), short interfering RNAs (siRNAs) and microRNAs (miRNAs), long RNAs like messenger RNAs (mRNA), or even double-stranded DNA (dsDNA). Therefore, EVs are emerging as a potential platform for delivery of therapeutic molecules.

The inventors have previously harnessed red blood cell-derived EVs (RBCEVs) to deliver therapeutic reagents including nucleic acids. This platform offers many advantages. Firstly, RBCEVs originate from enucleated red blood cells therefore they contain little or no DNA. Secondly, the production of RBCEVs can be done in a larger scale as compared to other sources of EVs due to the ease of blood collection and the abundance of RBCs in the blood. Thirdly, they can be delivered autologously or allogeneically similar to blood transfusion with matching blood types. RBCEVs have been shown to provide a robust delivery of therapeutic RNAs including antisense oligonucleotides (ASOs), mRNA and guide RNA (gRNAs) to treat cancer in mouse models, see e.g. PCT/SG2018/050596.

The present invention has been devised in light of the above considerations.

### SUMMARY OF THE INVENTION

The inventors have discovered that RBCEVs may be used to deliver nucleic acids to naïve T cells effectively without inducing cell death or activation. RBCEVs are taken up robustly by T cells and RBCEVs can be used to load nucleic acid into T cells. Thus, RBCEVs can be used to deliver therapeutic nucleic acid for genetic modification of T cells for ex vivo or in vivo immunotherapies.

Methods disclosed herein involve a step of contacting an immune cell with an RBCEV for sufficient time, and under conditions suitable for the immune cell to take up the RBCEV.

In one aspect, the present disclosure provides a method for delivering a nucleic acid into an immune cell, the method comprising incubating the immune cell with a red blood cell extracellular vesicle (RBCEV) loaded with a nucleic acid cargo.

In another aspect, the disclosure provides a method of transducing an immune cell, the method comprising incubating the immune cell with a RBCEV loaded with a nucleic acid cargo.

The immune cell may be a mononuclear cell, such as a peripheral blood mononuclear cell (PBMC). The immune cell is preferably a CD3+ cell. The immune cell may be a T cell.

In some cases, the immune cell is a dendritic cell. In some cases, the immune cell is a macrophage.

Preferably, the method is an in vitro or ex vivo method. Preferably, the immune cell is contacted with the RBCEV in vitro or ex vivo. In some cases, the immune cell is contacted with the RBCEV in vivo.

Methods disclosed herein may involve a step of loading an RBCEV with a nucleic acid cargo. In other methods, the RBCEV is provided with the nucleic acid cargo already loaded, such that the method does not require a step of loading the RBCEV with a nucleic acid cargo.

The nucleic acid cargo may comprise of RNA or DNA. For example, the nucleic acid cargo may comprise an antisense oligonucleotide, a messenger RNA, a siRNA, a miRNA, or a plasmid.

The method may involve a step of isolating an immune cell from a subject. In other methods, the method is performed on an immune cell that has been previously obtained from a subject. The method may involve administering an immune cell prepared by the methods disclosed herein to a subject. For example administering an immune cell loaded with nucleic acid or transduced, using an RBCEV as disclosed herein.

In another aspect, the disclosure provides the use of an RBCEV loaded with a nucleic acid for delivering a nucleic acid to an immune cell.

In another aspect, the disclosure provides an immune cell or a composition comprising a plurality of immune cells, wherein the immune cell(s) comprise an exogenous nucleic acid, wherein the nucleic acid is, or has been, delivered using a RBCEV. In some cases, the immune cell is naïve, undifferentiated or non-activated.

In another aspect, the disclosure provides a method of treatment, the method comprising administering to a subject in need thereof a therapeutically effective amount of an immune cell as disclosed herein, or an immune cell disclosed herein for use in such a method, or for the manufacture of a medicament for use in the treatment of a disease or disorder. In another aspect, the disclosure provides a method of treatment, the method comprising administering to an immune cell of a subject a therapeutically effective amount of an RBCEV loaded with a cargo, or an RBCEV for use in such a method, or for the manufacture of a medicament for use in the treatment of a disease or disorder.

### SUMMARY OF THE FIGURES

Embodiments and experiments illustrating the principles of the invention will be discussed with reference to the accompanying figures in which:

FIG. 1. Mouse T cells readily take up human RBCEVs. (A) Schematic representation of the uptake assay by labelling RBCEVs with CFSE dye. (B) Flow cytometry analysis of CFSE-labelled RBCEVs uptake by mouse CD3+ T cells. (C) Percentage of mouse CD3+ T cells bearing activation makers CD44 and CD69 when the cells were incubated with or without RBCEVs (n=3 replicates). (D) Schematic representation of antisense oligonucleotides (ASOs) delivered to mouse CD3+ mouse T cells via RBCEVs. After 24 h and 72h, cells were collected for qPCR and flow cytometry, respectively. (E) Normalized levels of miR-125b relative to internal control (snoRNA234) in CD3+ T cells treated as in (D) (n=3 replicates). Bar graphs represent mean±SEM.*** p<0.001 determined by Student's t-test.

FIG. 2. Human PBMCs take up RBCEVs loaded with antisense oligonucleotides (ASO). (A) Schematic representation of the uptake assay including the incubation of human PBMCs with RBCEVs that were labeled with CFSE. (B) Flow cytometry analysis of CFSE-labelled RBCEV uptake by total PBMCs and by each subpopulation. (C) Schematic representation of the uptake assay including incubation of human PBMCs with RBCEVs that were loaded with a Cy5-labeled control ASO (Cy5-NC-ASO). (D) Flow cytometry analysis of Cy5 fluorescent signals in PBMCs incubated with Cy5-NC-ASO-loaded RBCEVs compared to untreated and treatments with unloaded ASO.

FIG. 3. Comparison of ASO delivery by RBCEVs with other methods of transfection in human lymphocytes. (A) Schematic representation of transfection protocols used to deliver FAM-NC-ASO into human CD8 T cells. (B) Flow cytometry analysis of FAM signal in human CD8 T cells transfected with FAM-NC-ASO by different methods at 24 and 120h time points. (C) Percentage of viable CD8 T cells transfected with FAM-NC-ASO by different methods at 120h time point (n=3 replicates). Bar graphs represent mean±SEM. ** p<0.01, *** p<0.001 determined by Student's t-test.

FIG. 4. Transfection of ASOs and miRNA mimics into human CD8 T cells can suppress or upregulate microRNA expression. (A) Schematic representation of the transfection assay by electroporating RBCEVs with either ASOs or mimics then incubating with human CD8 T cells. After 72h, cells were collected for qRT-PCR. (B, C) Levels of miR-29a and its targets in adult CD8 T cells (n=3 replicates) transfected with miR-29a ASOs (29-ASO) or negative control ASOs (NC-ASO). (D, E) Levels of miR-29a and its targets in cord blood CD8 T cells (n=3 replicates) transfected with miR-29a mimics (29 mimic) or NC mimics (NC mimic). Bar graphs represent mean±SEM. * p<0.05, *** p<0.001 determined by Student's t-test.

FIG. 5. Transfected CD8 T cells show changes of miRNA targets without significant activation and differentiation. (A) Schematic representation of the transfection assay by electroporating RBCEVs with either ASOs or mimics then incubating with human CD8 T cells. After 120h, cells were collected for flow cytometry. (B) Relative geometry mean fluorescent intensity (gMFI) of miR-29a targets (EOMES and TBET) in naive adult CD8 T cells (n=4 replicates) transfected with 29-ASO or NC-ASO. (C) Relative percentage of activation (CD44NEG) and differentiation (CD62LPOS) of naive adult CD8 T cells (n=4 replicates) treated as in (B). (D) Relative gMFI of miR-29a targets in naive cord blood CD8 T cells (n=4 replicates) transfected with 29 mimic or NC mimic. (E) Relative activation and differentiation of naive cord blood CD8 T cells (n=4 replicates) treated as in (D). Bar graphs represent mean±SEM. *** p<0.001 determined by Student's t-test.

FIG. 6. Transfected T lymphocytes express mCherry without changes in viability. (A) Schematic representation of the transfection assay by loading RBCEVs with transfection reagent (TR) and mRNA. After 24h, cells were collected for flow cytometry. (B) Representative histogram of mCherry signals in human T lymphocytes, CD4 and CD8 T cells treated as in (A). (C) Relative gMFI of mCherry signals in different cell populations (n=3 replicates) of human lymphocytes treated as in (A). (D) Percentage of viable cells after transfection in different treatment groups. Bar graphs represent mean±SEM. * p<0.05 determined by Student's t-test.

FIG. 7. Transfected T lymphocytes express GFP without changes in viability. (A) Schematic representation of the transfection assay by loading RBCEVs with transfection reagent (TR) and plasmid (MC-GFP). After 72h, cells were collected for flow cytometry. (B) Representative histogram of GFP signals in human CD3+ T lymphocytes treated as in (A). (C) Relative gMFI of GFP signals in different treatments (n=4 replicates) of human lymphocytes treated as in (A). (D) Percentage of viable cells after transfection in different groups treated as in (A). Bar graphs represent mean±SEM. * p<0.05 determined by Student's t-test.

FIG. 8. Transfected T lymphocytes express cas9 proteins. (A) Schematic representation of the transfection assay by loading RBCEVs with transfection reagent (TR) and HA-tagged Cas9 mRNA. After 24 and 48h, cells were collected for immunostaining. (B) Representative immunostainings of Cas9 proteins in human CD8+ T lymphocytes treated as in (A). (C) Percentage of Cas9 positive CD8 T cells treated as in (A) at 24 and 48h. (D) Representative immunostaining images of Cas9 proteins in human CD4+ T lymphocytes treated as in (A). (E) Percentage of Cas9 positive CD4 T cells treated as in (A) at 24 and 48h. Images were taken at 20× magnification.

FIG. 9. RBCEVs are taken up by human dendritic cells. (A) FACS analysis of dendritic cell maturation from monocytes, based on the expression of activation marker HLA-DR and co-stimulatory markers CD80 and CD86. (B) Uptake of 0.1 or 0.2 mg CFSE labelled RBCEVs by monocyte-derived dendritic cells after 24 or 48 hours of incubation.

FIG. 10. Delivery of mRNA to mouse splenocytes using RBCEVs. FACS analysis of mCherry in mouse total splenocytes, conventional dendritic cells type 1 (cDC1) and 2 (cDC2), NK cells and CD8+ T cell and CD4+ T cell after an incubation with mCherry mRNA loaded RBCEVs for 48 hours. In this experiment, RBCEVs were loaded with mCherry mRNA and incubated with splenocytes from immunocompetent C57BL/6 mice. Cells were gate based on CD45+ then cell-type specific markers as indicated.

FIG. 11. Delivery of plasmid DNA to mouse splenocytes using RBCEVs. Expression of GFP in mouse total splenocytes, conventional dendritic cells type 1 (cDC1) and 2 (cDC2), NK cells, CD8+ T cell and CD4+ T cells after an incubation with GFP-minicircles (MC) loaded RBCEVs for 48 hours.

FIG. 12. Genome editing using RNA loaded RBCEVs. (A) Schematic representation of miR-125b knock-out in activated human CD3 T cells. Cas9 mRNA and sgRNAs (1:1 w/w) targeting miR-125b or RAB11a (control) were loaded separately by TR to RBCEVs. After the last wash, the EVs were pooled together and incubated with the T cells at different dosages (1 or 2 μg based on the sgRNA amounts). Cells were collected for qRT-PCR 24h post transfection. (B) miR-125b levels in T cells treated as in (C) (n=3). Bar graphs represent mean±SEM. * p<0.05, ** p<0.01, n.s. not significant, determined by Student's Ttest.

FIG. 13. Delivery of mRNA into mouse bone marrow-derived dendritic cells (BMDCs) via RBCEVs. (A) Schematic representation of RNA delivery to BMDCs by loading RBCEVs with mCherry mRNA and incubating BMDCs with RNA loaded RBCEVs. After 24h, cells were collected for flow cytometry. (B) Flow cytometry analysis of maturation markers for conventional dendritic cells type 1 (cDC1s) and plasmacytoid dendritic cells (pDCs) after differentiation of BM cells for 15 days. (C) Flow cytometry analysis of mCherry in mouse BMDCs and their subtypes treated with mCherry-mRNA-loaded RBCEVs.

FIG. 14. Delivery of RNA loaded RBCEVs to immune cells in the lung. (A) Schematic representation of the pulmonary delivery in C57BL/6 mice. AF647 labeled nontargeting siRNA (AF647-RNA) were loaded onto RBCEVs using a transfection reagent (TR). The loaded EVs were delivered to the the lung using an aerosolizer. After 24h, mice were sacrificed and lung cells were dissociated for a flow cytometry analysis of different immune cells within the lungs. (B) Representative dot plots of AF647-RNA signals in total lung cells of mice treated as in (A) including untreated, flowthrough, and RNA loaded RBCEVs (RNA+TR+EVs). (C) Representative histograms of AF647-RNA signals in different immune cell types (macrophages, dendritic cells, neutrophils, and alveolar macrophages) in mouse lungs treated as in (A).

### DETAILED DESCRIPTION OF THE INVENTION

The present invention relates to RBCEVs and their use to deliver therapeutic nucleic acids for genetic modification of immune cells for ex vivo or in vivo immunotherapies, or for in vitro transfection for e.g. gene expression studies. Delivery of therapeutic molecules into immune cells using RBCEVs is efficient, simple and cost effective.

The inventors have found that RBCEVs are taken up robustly by T cells and can deliver stable nucleic acid that functions well in the transfected cells. RBCEVs do not contain oncogenic nucleic acid or growth factors that are usually abundant in EVs from cancer cells or stem cells, meaning that RBCEVs do not pose transformation risk to recipient cells. In addition, DNA delivered by RBCEVs does not integrate into the genome of the cell, so there is negligible risk of insertional mutagenesis.

Advantageously, nucleic acids can be delivered to naïve T cells by RBCEVs without causing cell death, activation or differentiation of the naïve cells.

The cost of RBCEV production is low because RBCs are abundant in human blood, which can be obtained easily, and a large number of EVs can be purified from RBCs without having to culture the cells. RBCEVs are also likely to be non-immunogenic, with matched blood groups, unlike viruses and most synthetic transfection reagents.

Thus, the use of RBCEVs to transduce immune cells is safer and more efficient than viral-based or other non-viral-based methods. The present invention provides methods for delivering nucleic acid to immune cells using RBCEVs.

### Extracellular Vesicles

The term “extracellular vesicle” (EV) as used herein refers to a small vesicle-like structure released from a cell into the extracellular environment. In particularly preferred aspects disclosed herein, the extracellular vesicles are derived from red blood cells (RBCEVs).

Extracellular vesicles (EVs) are substantially spherical fragments of plasma membrane or endosomal membrane between 50 and 1000 nm in diameter. Extracellular vesicles are released from various cell types under both pathological and physiological conditions. Extracellular vesicles have a membrane. The membrane may be a double layer membrane (i.e. a lipid bilayer). The membrane may originate from the plasma membrane. Accordingly, the membrane of the extracellular vesicle may have a similar composition to the cell from which it is derived. In some aspects disclosed herein, the extracellular vesicles are substantially transparent.

The term extracellular vesicles encompasses exosomes, microvesicles, membrane microparticles, ectosomes, blebs and apoptotic bodies. Extracellular vesicles may be produced via outward budding and fission of cellular membrane. The production may be a natural process, or a chemically induced or enhanced process. In some aspects disclosed herein, the extracellular vesicle is a microvesicle produced via chemical induction.

Extracellular vesicles may be classified as exosomes, microvesicles or apoptotic bodies, based on their origin of formation. Microvesicles are a particularly preferred class of extracellular vesicle according to the invention disclosed herein. Preferably, the extracellular vesicles of the invention have been shed from the plasma membrane, and do not originate from the endosomal system. In certain aspects described herein, the extracellular vesicles are not exosomes. In some cases the extracellular vesicles are non-exosomal EVs.

In some aspects and embodiments of the present disclosure the extracellular vesicle is not an exosome. In some aspects and embodiments of the present disclosure the extracellular vesicle is not an ectosome. In some aspects and embodiments of the present disclosure the extracellular vesicle is not a bleb. In some aspects and embodiments of the present disclosure the extracellular vesicle is not an apoptotic body.

In some aspects and embodiments of the present disclosure the extracellular vesicle is a microvesicle or a membrane microparticle.

Extracellular vesicles disclosed herein may be derived from various cells, such as red blood cells, white blood cells, cancer cells, stem cells, dendritic cells, macrophages and the like. In a preferred embodiment, the extracellular vesicles are derived from a red blood cell, although extracellular vesicles from any source may be used, such as from cell lines. In preferred aspects described herein, the extracellular vesicles are derived from red blood cells.

Microvesicles or microparticles arise through direct outward budding and fission of the plasma membrane. Microvesicles are typically larger than exosomes, having diameters ranging from 100-500 nm. In some cases, a composition of microvesicles comprises microvesicles with diameters ranging from 50-1000 nm, from 50-750 nm, from 50-500 nm, from 50-300 nm, from 50-200 nm, from 50-150 nm, from 101-1000 nm, from 101-750 nm, from 101-500 nm, from 101-300 nm, from 100-300 nm, or from 100-200 nm. Preferably, the diameters are from 100-300 nm.

A population of microvesicles, for example as present in a composition, pharmaceutical composition, medicament or preparation, will comprise microvesicles with a range of different diameters, the median diameter of microvesicles within a microvesicle sample can range 50-1000 nm, from 50-750 nm, from 50-500 nm, from 50-300 nm, from 50-200 nm, from 50-150 nm, from 101-1000 nm, from 101-750 nm, from 101-500 nm, from 101-300 nm, from 100-300 nm, from 100-200 nm, or from 100-150 nm. Preferably, the median diameter is in one of the ranges: 50-300 nm, 50-200 nm, 50-150 nm, 100-300 nm, 100-200 nm, or 100-150 nm. The mean average diameter may be one of 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, optionally ±1, 2, 3, 4, 5, 6, 7, 8, 9 or 10 nm.

The diameter of exosomes ranges from around 30 to around 100 nm. In some cases, a population of exosomes, as may be present in a composition, comprises exosomes with diameters ranging from 10-200 nm, from 10-150 nm, from 10-120 nm, from 10-100 nm, from 20-150 nm, from 20-120 nm, from 25-110 nm, from 25-100 nm, or from 30-100 nm. Preferably, the diameters are from 30-100 nm. A population of exosomes, for example as present in a composition, pharmaceutical composition, medicament or preparation, will comprise exosomes with a range of different diameters, the median diameter of exosomes within a sample can range ranging from 10-200 nm, from 10-150 nm, from 10-120 nm, from 10-100 nm, from 20-150 nm, from 20-120 nm, from 25-110 nm, from 25-100 nm, or from 30-100 nm. Preferably, the median diameter is between 30-100 nm. The mean average diameter may be one of 10 nm, 20 nm, 30 nm, 40 nm, 50 nm, 60 nm, 70 nm, 80 nm, 90 nm, 100 nm, 110 nm, or 120 nm, optionally ±1, 2, 3, 4, 5, 6, 7, 8, 9 or 10 nm.

A population of extracellular vesicles may comprise one of at least 10, 100, 1000, 10⁴, 10⁵, 10⁶, 10⁷, 10⁸, 10⁹, 10¹⁰, 10¹¹, 10¹², 10¹³ or 10¹⁴ extracellular vesicles (optionally per ml of carrier).

Exosomes are observed in a variety of cultured cells including lymphocytes, dendritic cells, cytotoxic T cells, mast cells, neurons, oligodendrocytes, Schwann cells, and intestinal epithelial cells. Exosomes originate from the endosomal network that locates in within multivesicular bodies, large sacs in the cytoplasm. These sacs fuse to the plasma membrane, before being released into extracellular environment.

Apoptotic bodies or blebs are the largest extracellular vesicles, ranging from 1-5 μm. Nucleated cells undergoing apoptosis pass through several stages, beginning with condensation of the nuclear chromatin, membrane blebbing and finally release of EVs including apoptotic bodies.

Preferably, the extracellular vesicles are derived from human cells, or cells of human origin. The extracellular vesicles of the invention may have been induced from cells contacted with a vesicle inducing agent. The vesicle inducing agent may be calcium ionophore, lysophosphatidic acid (LPA), or phorbol-12-myristat-13-acetate (PMA).

In many aspects described herein, the cells are not modified. In particular, the cells from which the extracellular vesicles are derived do not comprise exogenous nucleic acid or proteins. In some cases, the cells are ex vivo, such as resulting from a blood draw. In some cases, the cells have not been modified, such as transduced, transfected, infected, or otherwise modified, but are substantially unchanged as compared to the cells in vivo. Where the cells are red blood cells, the cells may contain no DNA, or may contain substantially no DNA. The red blood cells may be DNA free. Accordingly, in preferred embodiments the extracellular vesicles are loaded with their nucleic acid cargo after the extracellular vesicles have been formed and isolated. Preferably, the extracellular vesicles do not contain nucleic acid, particularly DNA, that was present in the cells from which they are derived.

### Red Blood Cell Extracellular Vesicles (RBCEVs)

In certain aspects disclosed herein, the extracellular vesicles are derived from red blood cells (erythrocytes). Red blood cells are a good source of EVs for a number of reasons. Because red blood cells are enucleated, RBCEVs contain less nucleic acid than EVs from other sources. RBCEVs do not contain endogenous DNA. RBCEVs may contain miRNAs or other RNAs. RBCEVs are free from oncogenic substances such as oncogenic DNA or DNA mutations.

In some cases, the EVs are non-exosomal EVs derived from red blood cells, e.g. human red blood cells.

In some cases, the RBCEVs are isolated from RBCs. A method for isolation and characterisation of RBCEVs is described in Usman et al. (Efficient RNA drug delivery using red blood cell extracellular vesicles. Nature Communications 9, 2359 (2018) doi:10.1038/s41467-018-04791-8), incorporated herein in its entirety by reference.

RBCEVs may comprise haemoglobin and/or stomatin and/or flotillin-2. They may be red in colour. Typically RBCEVs exhibit a domed (concave) surface, or “cup shape” under transmission electron microscopes. The RBCEV may be characterised by having cell surface CD235a.

RBCEVs according to the invention may be about 100 nm to about 300 nm in diameter. In some cases, a composition of RBCEVs comprises RBCEVs with diameters ranging from 50-1000 nm, from 50-750 nm, from 50-500 nm, from 50-300 nm, from 50-200 nm, from 50-150 nm, from 101-1000 nm, from 101-750 nm, from 101-500 nm, from 101-300 nm, from 100-300 nm, from 100-200 nm or from 100-150 nm. Preferably, the diameters are from 50-300 nm, from 50-200 nm, from 50-150 nm, 100-300 nm, from 100-200 nm, or from 100-150 nm.

A population of RBCEVs, e.g. as may be present in a composition, will comprise RBCEVs with a range of different diameters, the median diameter of RBCEVs within a RBCEV sample can range from 50-1000 nm, from 50-750 nm, from 50-500 nm, from 50-300 nm, from 50-200 nm, from 50-150 nm, from 101-1000 nm, from 101-750 nm, from 101-500 nm, from 101-300 nm, from 100-300 nm, from 100-200 nm or from 100-150 nm. Preferably, the median diameter is between 50-300 nm, from 50-200 nm, from 50-150 nm, 100-300 nm, from 100-200 nm, or from 100-150 nm. The mean average diameter may be one of 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, optionally ±1, 2, 3, 4, 5, 6, 7, 8, 9 or 10 nm.

Preferably, the RBCEVs are derived from a human or animal blood sample or red blood cells derived from primary cells or immobilized red blood cell lines. The blood cells may be type matched to the patient to be treated, and thus the blood cells may be Group A, Group B, Group AB or Group O. Preferably the blood is Group O. The blood may be rhesus positive or rhesus negative. In some cases, the blood is Group O and/or rhesus negative, such as Type O-. The blood may have been determined to be free from disease or disorder, such as free from HIV, sickle cell anaemia, malaria. However, any blood type may be used. In some cases, the RBCEVs are autologous and derived from a blood sample obtained from the patient to be treated. In some cases, the RBCEVs are allogenic and not derived from a blood sample obtained from the patient to be treated.

RBCEVs may be isolated from a sample of red blood cells. Protocols for obtaining EVs from red blood cells are known in the art, for example in Danesh et al. (2014) Blood. 2014 Jan. 30; 123(5): 687-696. Methods useful for obtaining EVs may include the step of providing or obtaining a sample comprising red blood cells, inducing the red blood cells to produce extracellular vesicles, and isolating the extracellular vesicles. The sample may be a whole blood sample. Preferably, cells other than red blood cells have been removed from the sample, such that the cellular component of the sample is red blood cells.

The red blood cells in the sample may be concentrated, or partitioned from other components of a whole blood sample, such as white blood cells and plasma. Red blood cells may be concentrated by centrifugation. The sample may be subjected to leukocyte reduction.

The sample comprising red blood cells may comprise substantially only red blood cells. Extracellular vesicles may be induced from the red blood cells by contacting the red blood cells with a vesicle inducing agent. The vesicle inducing agent may be calcium ionophore, lysophosphatidic acid (LPA), or phorbol-12-myristat-13-acetate (PMA).

RBCEVs may be isolated by centrifugation (with or without ultracentrifugation), precipitation, filtration processes such as tangential flow filtration, or size exclusion chromatography (e.g. see Usman et al., supra). In this way, RBCEVs may be separated from RBCs and other components of the mixture.

Extracellular vesicles may be obtained from red blood cells by a method comprising: obtaining a sample of red blood cells; contacting the red blood cells with a vesicle inducing agent; and isolating the induced extracellular vesicles.

The red blood cells may be separated from a whole blood sample containing white blood cells and plasma by low speed centrifugation and using leukodepletion filters. In some cases, the red blood cell sample contains no other cell types, such as white blood cells. In other words, the red blood cell sample consists substantially of red blood cells. The red blood cells may be diluted in buffer such as PBS prior to contacting with the vesicle inducing agent. The vesicle inducing agent may be calcium ionophore, lysophosphatidic acid (LPA) or phorbol-12-myristat-13-acetate (PMA). The vesicle inducing agent may be about 10 nM calcium ionophore. The red blood cells may be contacted with the vesicle inducing agent overnight, or for at least 1, at least 2, at least 3, at least 4, at least 5, at least 6, at least 7, at least 8, at least 9, at least 10, at least 11, at least 12 or more than 12 hours. The mixture may be subjected to low speed centrifugation to remove RBCs, cell debris, or other non-RBCEVs matter and/or passing the supernatant through an about 0.45 μm syringe filter. RBCEVs may be concentrated by ultracentrifugation, such as centrifugation at around 100,000×g. The RBCEVs may be concentrated by ultracentrifugation for at least 10 minutes, at least 20 minutes, at least 30 minutes, at least 40 minutes, at least 50 minutes or at least one hour. The concentrated RBCEVs may be suspended in cold PBS. They may be layered on a 60% sucrose cushion. The sucrose cushion may comprise frozen 60% sucrose. The RBCEVs layered on the sucrose cushion may be subject to ultracentrugation at 100,000×g for at least one hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours or more. Preferably, the RBCEVs layered on the sucrose cushion may be subject to ultracentrifugation at 100,000×g for about 16 hours. The red layer above the sucrose cushion is then collected, thereby obtaining RBCEVs. The obtained RBCEVs may be subject to further processing, such as washing, tagging, and optionally loading.

### Surface Tagging

Extracellular vesicles may comprise a tag, preferably attached to, or inserted through, the vesicle membrane.

The extracellular vesicles may have, at their surface, a tag. The tag is preferably a protein or peptide sequence. The tag may be a peptide or protein. It may be a modified peptide or protein, such as a glycosylated or biotinylated protein or peptide. The tag may be covalently linked to the extracellular vesicle, such as covalently linked to a membrane protein in the extracellular vesicle. The tag may have been added to the extracellular vesicle after the extracellular vesicle had formed. The tag may be linked to the extracellular vesicle by a sequence that comprises or consists of a sequence that is, or that is derived from, a protein ligase recognition sequence. For example, the tag may be linked to the extracellular vesicle by a sequence that comprises 100% sequence identity to a protein ligase recognition sequence, or about 90%, about 80%, about 70%, about 60%, about 50% or about 40% sequence identity to a protein ligase recognition sequence. The amino acid sequence may comprise LPXT.

The tag may be presented on the external surface of the vesicle, and is thus exposed to the extravesicular environment.

The tag may be an exogenous molecule. In other words, the tag is a molecule that is not present on the external surface of the vesicle in nature. In some cases, the tag is an exogenous molecule that is not present in the cell or red blood cell from which the extracellular vesicle is derived.

The tag may increase the stability, uptake efficiency and availability in the circulation of the extracellular vesicles.

In some cases, the tag acts to present the extracellular vesicles and extracellular vesicles containing cargoes in the circulation and organs in the body. The peptides and proteins can act as therapeutic molecules such as blocking/activating target cell function or presenting antigens for vaccination. They can also act as probes for biomarker detection such as diagnosis of toxins.

The tag may contain a functional domain and a protein ligase recognition sequence. The functional domain may be capable of binding to a target moiety, capable of detection, or capable of inducing a therapeutic effect. The functional domain may be capable of binding to a target molecule. Tags comprising such a functional domain may be referred to herein as binding molecules. A binding molecule is one that is capable of interacting specifically with a target molecule. Extracellular vesicles comprising a binding moiety may be particularly useful for delivering a cargo or a therapeutic agent to a cell that has the target molecule. Suitable binding molecules include antibodies and antigen binding fragments (sometimes known as antibody fragments), ligand molecules and receptor molecules. The binding molecule will bind to a target of interest. The target may be a molecule associated with, such as expressed on the surface of, a cell of interest. The ligand may form a complex with a biomolecule on the target cell, such as a receptor molecule. The target may be a molecule associated with an immune cell, such as a cell surface marker.

Suitable binding molecules include antibodies and antigen binding fragments. Fragments, such as Fab and Fab2 fragments may be used as can genetically engineered antibodies and antibody fragments. The variable heavy (VH) and variable light (VL) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent parented antibody (Morrison et al (1984) Proc. Natl. Acad. Sd. USA 81, 6851-6855). Antibodies or antigen binding fragments useful in the extracellular vesicles disclosed herein will recognise and/or bind to, a target molecule.

That antigenic specificity is conferred by variable domains and is independent of the constant domains is known from experiments involving the bacterial expression of antibody fragments, all containing one or more variable domains. These molecules include Fab-like molecules (Better et al. (1988) Science 240, 1041); Fv molecules (Skerra et al. (1988) Science 240, 1038); single-chain Fv (ScFv) molecules where the VH and VL partner domains are linked via a flexible oligopeptide (Bird et al. (1988) Science 242, 423; Huston et al. (1988) Proc. Natl. Acad. Sd. USA 85, 5879) and single domain antibodies (dAbs) comprising isolated V domains (Ward et al. (1989) Nature 341, 544). A general review of the techniques involved in the synthesis of antibody fragments which retain their specific binding sites is to be found in Winter & Milstein (1991) Nature 349, 293-299. Antibodies and fragments useful herein may be human or humanized, murine, camelid, chimeric, or from any other suitable source.

By “ScFv molecules” we mean molecules wherein the VH and VL partner domains are covalently linked, e.g. directly, by a peptide or by a flexible oligopeptide. Fab, Fv, ScFv and sdAb antibody fragments can all be expressed in and secreted from E. coli, thus allowing the facile production of large amounts of the said fragments.

Whole antibodies, and F(ab′)2 fragments are “bivalent”. By “bivalent” we mean that the said antibodies and F(ab′)2 fragments have two antigen combining sites. In contrast, Fab, Fv, ScFv and sdAb fragments are monovalent, having only one antigen combining site. Monovalent antibody fragments are particularly useful as tags, because of their small size.

A preferred binding molecule may be a sdAb. By “sdAb” we mean single domain antibody consisting of one, two or more single monomeric variable antibody domains. sdAb molecules are sometimes referred to as dAb.

In some cases, the binding molecule is a single chain antibody, or scAb. A scAb consists of covalently linked VH and VL partner domains (e.g. directly, by a peptide, or by a flexible oligopeptide) and optionally a light chain constant domain.

Other suitable binding molecules include ligands and receptors that have affinity for a target molecule. The tag may be a ligand of a cell surface receptor. Examples include streptavidin and biotin, avidin and biotin, or ligands of other receptors, such as fibronectin and integrin. The small size of biotin results in little to no effect to the biological activity of bound molecules. As biotin and streptavidin, biotin and avidin, and fibronectin and integrin bind their pairs with high affinity and specificity, they are very useful as binding molecules. The Avidin-biotin complex is the strongest known non-covalent interaction (Kd=10-15M) between a protein and ligand. Bond formation is rapid, and once formed, is unaffected by extremes of pH, temperature, organic solvents and other denaturing agents. The binding of biotin to streptavidin and is also strong, rapid to form and useful in biotechnology applications.

The functional domain may comprise or consist of a therapeutic agent. The therapeutic agent may be an enzyme. It may be an apoptotic inducer or inhibitor.

The functional domain may comprise an antigen or antibody recognition sequence. The tag may comprise one or more short peptides derived from one or more antigenic peptides. The peptide may be a fragment of an antigenic peptide. Suitable antigenic peptides are known to one of skill in the art.

The functional domain may comprise or consist of a detectable moiety. Detectable moieties include fluorescent labels, colorimetric labels, photochromic compounds, magnetic particles or other chemical labels. The detectable moiety may be biotin or a His tag.

The tag may comprise a spacer or linker moiety. The spacer or linker may be arranged between the tag and the protein ligase recognition sequence. The spacer or linker may be linked to the N or C terminus of the tag. The spacer or linker may be arranged so as not to interfere or impede the function of the tag, such as the target binding activity by the tag. The spacer or linker may be a peptide sequence. In some case, the spacer or linker is a series of at least 1, at least 2, at least 3, at least 4, at least 5, at least 6, at least 7, at least 8, at least 9, at least 10 amino acids, at least 11 amino acids, at least 12 amino acids, at least 13 amino acids, at least 14 amino acids or at least 15 amino acids. The spacer or linker may be flexible. The spacer may comprise a plurality of glycine and/or serine amino acids.

Spacer and linker sequences are known to the skilled person, and are described, for example in Chen et al., Adv Drug Deliv Rev (2013) 65(10): 1357-1369, which is hereby incorporated by reference in its entirety. In some embodiments, a linker sequence may be a flexible linker sequence. Flexible linker sequences allow for relative movement of the amino acid sequences which are linked by the linker sequence. Flexible linkers are known to the skilled person, and several are identified in Chen et al., Adv Drug Deliv Rev (2013) 65(10): 1357-1369. Flexible linker sequences often comprise high proportions of glycine and/or serine residues.

In some cases, the spacer or linker sequence comprises at least one glycine residue and/or at least one serine residue. In some embodiments the linker sequence consists of glycine and serine residues. In some cases, the spacer or linker sequence has a length of 1-2, 1-3, 1-4, 1-5 or 1-10 amino acids.

Inclusion of the spacer or linker may improve the efficiency of the protein ligase reaction between the extracellular vesicle and the tag moiety. The term “tag” as used herein may encompass a peptide comprising a tag, a spacer, and protein ligase recognition sequence.

Suitable protein ligase recognition sequences are known in the art. The protein ligase recognition sequence is recognised by the protein ligase used in the method of tagging the extracellular vesicles. For example, if the protein ligase used in the method is a sortase, then the protein ligase recognition sequence is a sortase binding site. In those cases, the sequence may be LPXTG (where X is any naturally occurring amino acid), preferably LPETG. Alternatively, where the enzyme is Asparaginyl endopeptidase 1 (AEP1), the protein ligase recognition sequence may be NGL. The protein ligase binding site may be arranged at the C terminus of the peptide or protein.

The tag may additionally comprise one or more further sequences to aid in purification or processing of the tag, during production of the tag itself, during the tagging method, or for subsequent purification. Any suitable sequence known in the art may be used. For example, the sequence may be an HA tag, a FLAG tag, a Myc tag, a His tag (such as a poly His tag, or a 6×His tag).

The tag may be linked to substantially all of the extracellular vesicles in a population or composition. Compositions disclosed herein may comprise extracellular vesicles in which at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, or at least 97% of the extracellular vesicles comprise the tag. Preferably, at least 85%, at least 90%, at least 95%, at least 96% or at least 97% of the extracellular vesicles comprise the tag. In some cases, different extracellular vesicles within the composition comprise different tags. In some cases, the extracellular vesicles comprise the same, or substantially the same, tag.

Methods for incorporating a tag are described in PCT/SG2019/050481, WO 2014/183071 A2, WO 2014/183066 A2 and US 2014/0030697 A1, each incorporated herein by reference in its entirety.

### Cargo

Extracellular vesicles described herein may be loaded with, or contain, a cargo. The present disclosure is particularly concerned with nucleic acid cargo. In some preferred embodiments the cargo comprises, or consists of, RNA or a chemically modified RNA. The term “cargo” is used interchangeably with “load” herein.

A nucleic acid cargo refers to a nucleic acid (e.g. oligonucleotide or polynucleotide) loaded into or onto an extracellular vesicle. A nucleic acid cargo normally refers to an oligonucleotide strand (which may be in any form, e.g. single stranded, double stranded, super-coiled or not super-coiled, chromosomal or non-chromosomal). The nucleic acid may be conjugated to, or complexed with, other molecules, e.g. carriers, stabilisers, histones, lipophilic agents.

Methods disclosed herein may be used for any nucleic acid cargo. Nucleic acid may be double or single stranded. Preferably, the nucleic acid is single stranded. The nucleic acid may be circular.

The cargo is preferably exogenous. In other words, the nucleic acid is not present in the extracellular vesicles when they are newly generated, and/or in the cells from which the extracellular vesicles are derived. The cargo may be synthetic, having been designed and/or constructed in vitro or in silico.

The cargo may be a therapeutic oligonucleotide or a diagnostic oligonucleotide. The cargo may exert a therapeutic effect in a target cell after being delivered to that target cell. The nucleic acid may encode a gene of interest. For example, the cargo may encode a functional gene to replace an absent gene, repair a defective gene, or induce a therapeutic effect in a target tissue. In some cases, the cargo is a reporter gene or encodes a molecule that is readily detectable.

In some cases, the cargo may be a nucleic acid. The nucleic acid may be single stranded or double stranded. The cargo may be an RNA. The RNA may be a therapeutic RNA. The RNA may be a small interfering RNA (siRNA), a messenger RNA (mRNA), a guide RNA (gRNA), a circular RNA, a microRNA (miRNA), a piwiRNA (piRNA), a transfer RNA (tRNA), or a long noncoding RNA (lncRNA) produced by chemical synthesis or in vitro transcription. In some cases, the cargo is an antisense oligonucleotide, for example, having a sequence that is complementary to an endogenous nucleic acid sequence such as a transcription factor, miRNA or other endogenous mRNA.

The cargo may be, or may encode, a molecule of interest. For example, the cargo may be an mRNA that encodes Cas9 or another nuclease. The cargo may encode one or more peptides/polypeptides of interest.

In some cases, the cargo is a nucleic acid that is, or that encodes, an siRNA or antisense oligonucleotide (ASO). Such cargo may be useful in methods of gene silencing or downregulating gene expression. The siRNA or ASO may correspond to a sequence that is expressed in a target cell, e.g. an mRNA sequence. It may act to inhibit or enhance the expression of a particular gene or protein of interest. The nucleic acid may encode an siRNA or ASO corresponding to a miRNA expressed in a target cell.

The cargo may comprise or encode an mRNA. The mRNA may encode a transgene.

In the cell, an antisense nucleic acid may hybridize to the corresponding mRNA, forming a double-stranded molecule. The antisense nucleic acids may interfere with the translation of the mRNA, since the cell will not translate an mRNA that is double-stranded. The use of antisense methods to inhibit the in vitro translation of genes is well known in the art (see e.g. Marcus-Sakura, Anal. Biochem. 1988, 172:289). Further, antisense molecules which bind directly to the DNA may be used. Antisense nucleic acids may be single or double stranded nucleic acids. Non-limiting examples of antisense nucleic acids include small interfering RNA (siRNA; including their derivatives or pre-cursors, such as nucleotide analogs), short hairpin RNAs (shRNA), micro RNAs (miRNA), saRNAs (small activating RNAs), small nucleolar RNAs (snoRNA) or certain of their derivatives or pre-cursors, long non-coding RNA (lncRNA), or single stranded molecules such as chimeric ASOs or gapmers. Antisense nucleic acid molecules may stimulate RNA interference (RNAi) or other cellular degradation mechanisms such as RNase degradation.

In some preferred embodiments, the cargo comprises or encodes an ASO that targets, e.g. hybridises to, a micro RNA. In some cases the ASO inhibits the function of the micro RNA and prevents the miRNA from post-transcriptionally regulating gene expression. In some cases the ASO functions to upregulate expression of one or more genes that are usually downregulated by a miRNA. Thus, an antisense nucleic acid cargo may interfere with transcription of target genes, interfere with translation of target mRNA and/or promote degradation of target mRNA. In some cases, an antisense nucleic acid is capable of inducing a reduction in expression of the target gene.

A “siRNA,” “small interfering RNA,” “small RNA,” or “RNAi” as provided herein, refers to a nucleic acid that forms a double stranded RNA, which double stranded RNA has the ability to reduce or inhibit expression of a gene or target gene when expressed in the same cell as the gene or target gene. The complementary portions of the nucleic acid that hybridize to form the double stranded molecule typically have substantial or complete identity. In one embodiment, a siRNA or RNAi is a nucleic acid that has substantial or complete identity to a target gene and forms a double stranded siRNA. In embodiments, the siRNA inhibits gene expression by interacting with a complementary cellular mRNA thereby interfering with the expression of the complementary mRNA. Typically, the nucleic acid is at least about 15-50 nucleotides in length (e.g., each complementary sequence of the double stranded siRNA is 15-50 nucleotides in length, and the double stranded siRNA is about 15-50 base pairs in length). In some embodiments, the length is 20-30 base nucleotides, preferably about 20-25 or about 24-29 nucleotides in length, e.g., 20, 21, 22, 23, 24, 25, 26, 27, 28, 29, or 30 nucleotides in length.

RNAi and siRNA are described in, for example, Dana et al., Int J Biomed Sci. 2017; 13(2): 48-57, herein incorporated by reference in its entirety. An antisense nucleic acid molecule may contain double-stranded RNA (dsRNA) or partially double-stranded RNA that is complementary to a target nucleic acid sequence. A double-stranded RNA molecule is formed by the complementary pairing between a first RNA portion and a second RNA portion within the molecule. The length of an RNA sequence (i.e. one portion) is generally less than 30 nucleotides in length (e.g. 29, 28, 27, 26, 25, 24, 23, 22, 21, 20, 19, 18, 17, 16, 15, 14, 13, 12, 11, 10 or fewer nucleotides). In some embodiments, the length of an RNA sequence is 18 to 24 nucleotides in length. In some siRNA molecules, the complementary first and second portions of the RNA molecule form the “stem” of a hairpin structure. The two portions can be joined by a linking sequence, which may form the “loop” in the hairpin structure. The linking sequence may vary in length and may be, for example, 5, 6, 7, 8, 9, 10, 11, 12, or 13 nucleotides in length. Suitable linking sequences are known in the art.

Suitable siRNA molecules for use in the methods of the present invention may be designed by schemes known in the art, see for example Elbashire et al., Nature, 2001 411:494-8; Amarzguioui et al., Biochem. Biophys. Res. Commun. 2004 316(4):1050-8; and Reynolds et al., Nat. Biotech. 2004, 22(3):326-30. Details for making siRNA molecules can be found in the websites of several commercial vendors such as Ambion, Dharmacon, GenScript, Invitrogen and OligoEngine. The sequence of any potential siRNA candidate generally can be checked for any possible matches to other nucleic acid sequences or polymorphisms of nucleic acid sequence using the BLAST alignment program (see the National Library of Medicine Internet website). Typically, a number of siRNAs are generated and screened to obtain an effective drug candidate, see, U.S. Pat. No. 7,078,196. siRNAs can be expressed from a vector and/or produced chemically or synthetically. Synthetic RNAi can be obtained from commercial sources, for example, Invitrogen (Carlsbad, Calif.). RNAi vectors can also be obtained from commercial sources, for example, Invitrogen.

The nucleic acid molecule may be, comprise, or encode a miRNA. The term “miRNA” is used in accordance with its plain ordinary meaning and refers to a small non-coding RNA molecule capable of post-transcriptionally regulating gene expression. In one embodiment, a miRNA is a nucleic acid that has substantial or complete identity to a target gene. In some embodiments, the miRNA inhibits gene expression by interacting with a complementary cellular mRNA thereby interfering with the expression of the complementary mRNA. Typically, the miRNA is at least about 15-50 nucleotides in length (e.g., each complementary sequence of the miRNA is 15-50 nucleotides in length, and the miRNA is about 15-50 base pairs in length). In some cases, the nucleic acid is synthetic or recombinant. The miRNA may be miR-29a. The miRNA may comprise or consist of the sequence 5′-ACUGAUUUCUUUUGGUGUUCAG-3′. In some cases the nucleic acid is a miRNA stem-loop.

Nucleic acids useful in the methods of the invention include antisense oligonucleotides, mRNA, or siRNAs that target oncogenic miRNAs (also known as oncomiRs) or transcription factors. The cargo may be a ribozyme or aptamer. In some cases, the nucleic acid is a plasmid.

In certain aspects described herein, the cargo is an antisense oligonucleotide (ASO). The antisense oligonucleotide may be complementary to a miRNA or mRNA. The antisense oligonucleotide comprises at least a portion which is complementary in sequence to a target mRNA sequence. The antisense oligonucleotide may bind to, and thereby inhibit, the target sequence. For example, the antisense oligonucleotide may inhibit the translation process of the target sequence. The miRNA may be a miRNA associated with cancer (Oncomir). The miRNA may be miR-125b.

In some aspects, the cargo is one or more components of a gene editing system. For example, a CRISPR/Cas9 gene editing system. For example, the cargo may include a nucleic acid which recognises a particular target sequence. The cargo may be a gRNA. Such gRNAs may be useful in CRISPR/Cas9 gene editing. The cargo may be a Cas9 mRNA or a plasmid encoding Cas9. The Cas9 enzyme may be substituted with a Cas12 or Cas 13 enzyme. Other gene editing molecules may be used as cargo, such as zinc finger nucleases (ZFNs) or Transcription activator-like effector nucleases (TALENs). The cargo may comprise a sequence engineered to target a particular nucleic acid sequence in a target cell. The gene editing molecule may specifically target a miRNA. For example, the gene editing molecule may be a gRNA that targets miR-125b. The gRNA may comprise or consist of the sequence 5′-CCUCACAAGUUAGGGUCUCA-3′.

In some embodiments the methods employ target gene editing using site-specific nucleases (SSNs). Gene editing using SSNs is reviewed e.g. in Eid and Mahfouz, Exp Mol Med. 2016 October; 48(10): e265, which is hereby incorporated by reference in its entirety. Enzymes capable of creating site-specific double strand breaks (DSBs) can be engineered to introduce DSBs to target nucleic acid sequence(s) of interest. DSBs may be repaired by either error-prone non-homologous end-joining (NHEJ), in which the two ends of the break are rejoined, often with insertion or deletion of nucleotides. Alternatively DSBs may be repaired by highly homology-directed repair (HDR), in which a DNA template with ends homologous to the break site is supplied and introduced at the site of the DSB.

SSNs capable of being engineered to generate target nucleic acid sequence-specific DSBs include ZFNs, TALENs and clustered regularly interspaced palindromic repeats/CRISPR-associated-9 (CRISPR/Cas9) systems.

ZFN systems are reviewed e.g. in Umov et al., Nat Rev Genet. (2010) 11(9):636-46, which is hereby incorporated by reference in its entirety. ZFNs comprise a programmable Zinc Finger DNA-binding domain and a DNA-cleaving domain (e.g. a FokI endonuclease domain). The DNA-binding domain may be identified by screening a Zinc Finger array capable of binding to the target nucleic acid sequence.

TALEN systems are reviewed e.g. in Mahfouz et al., Plant Biotechnol J. (2014) 12(8):1006-14, which is hereby incorporated by reference in its entirety. TALENs comprise a programmable DNA-binding TALE domain and a DNA-cleaving domain (e.g. a FokI endonuclease domain). TALEs comprise repeat domains consisting of repeats of 33-39 amino acids, which are identical except for two residues at positions 12 and 13 of each repeat which are repeat variable di-residues (RVDs). Each RVD determines binding of the repeat to a nucleotide in the target DNA sequence according to the following relationship: “HD” binds to C, “NI” binds to A, “NG” binds to T and “NN” or “NK” binds to G (Moscou and Bogdanove, Science (2009) 326(5959):1501.).

CRISPR is an abbreviation of Clustered Regularly Interspaced Short Palindromic Repeats. The term was first used at a time when the origin and function of these sequences were not known and they were assumed to be prokaryotic in origin. CRISPR are segments of DNA containing short, repetitive base sequences in a palindromic repeat (the sequence of nucleotides is the same in both directions). Each repetition is followed by short segments of spacer DNA from previous integration of foreign DNA from a virus or plasmid. Small clusters of CAS (CRISPR-associated) genes are located next to CRISPR sequences. RNA harboring the spacer sequence helps Cas (CRISPR-associated) proteins recognize and cut foreign pathogenic DNA. Other RNA-guided Cas proteins cut foreign RNA. A simple version of the CRISPR/Cas system, CRISPR/Cas9, has been modified to edit genomes. By delivering the Cas9 nuclease and a synthetic guide RNA (gRNA) into a cell, the cell's genome can be cut at a desired location, allowing existing genes to be removed and/or new ones added. CRISPR-Cas systems fall into two classes. Class 1 systems use a complex of multiple Cas proteins to degrade foreign nucleic acids. Class 2 systems use a single large Cas protein for the same purpose. Class 1 is divided into types I, Ill; and IV; class 2 is divided into types II, V, and VI. CRISPR genome editing uses a type H CRISPR system.

In some aspects, the EV is loaded with a CRISPR related cargo. In other words, the EV is useful in a method involving gene editing, such as therapeutic gene editing. In some cases, the EV is useful for in vitro gene editing. In some cases, the EV is useful for in vivo gene editing.

The cargo may be a guide RNA. The guide RNA may comprise a CRIPSR RNA (crRNA) and a trans-activating CRISPR RNA (tracrRNA). The crRNA contains a guide RNA that locates the correct section of host DNA along with a region that binds to tracrRNA forming an active complex. The tracrRNA binds to crRNA and forms the active complex. The gRNA combines both the tracrRNA and a crRNA, thereby encoding an active complex. The gRNA may comprise multiple crRNAs and tracrRNAs. The gRNA may be designed to bind to a sequence or gene of interest. The gRNA may target a gene for cleavage. Optionally, an optional section of DNA repair template is included. The repair template may be utilized in either non-homologous end joining (NHEJ) or homology directed repair (HDR).

The cargo may be a nuclease, such as a Cas9 nuclease. The nuclease is a protein whose active form is able to modify DNA. Nuclease variants are capable of single strand nicking, double strand break, DNA binding or other different functions. The nuclease recognises a DNA site, allowing for site specific DNA editing.

The gRNA and nuclease may be encoded on a plasmid. In other words, the EV cargo may comprise a plasmid that encodes both the gRNA and the nuclease. In some cases, an EV contains the gRNA and another EV contains or encodes the nuclease. In some cases, an EV contains a plasmid encoding the gRNA, and a plasmid encoding the nuclease. Thus, in some aspects, a composition is provided comprising EVs, wherein a portion of the EVs comprise or encode the nuclease such as Cas9, and a portion of the EVs comprise or encode the gRNA. In some cases, a composition containing EVs that comprise or encode the gRNA and a composition containing EVs that encode or contain the nuclease are co-administered. In some cases, the composition comprises EVS wherein the EVs contain an oligonucleotide that encodes both a gRNA and a nuclease.

CRISPR/Cas9 and related systems e.g. CRISPR/Cpf1, CRISPR/C2c1, CRISPR/C2c2 and CRISPR/C2c3 are reviewed e.g. in Nakade et al., Bioengineered (2017) 8(3):265-273, which is hereby incorporated by reference in its entirety. These systems comprise an endonuclease (e.g. Cas9, Cpf1 etc.) and the single-guide RNA (sgRNA) molecule. The sgRNA can be engineered to target endonuclease activity to nucleic acid sequences of interest.

In some cases, the nucleic acid cargo comprises one or more modified nucleotides or other modifications. Chemical modifications may include chemical substitution at a sugar position, a phosphate position, and/or a base position of the nucleic acid including, for example, incorporation of a modified nucleotide, incorporation of a capping moiety (e.g. 3′ capping), conjugation to a high molecular weight, non-immunogenic compound (e.g. polyethylene glycol (PEG)), conjugation to a lipophilic compound, substitutions in the phosphate backbone. For example, the nucleic acid may comprise one or more 2′-position sugar modifications, such as 2′-amino (2′-NH), 2′-fluoro (2′-F), and 2′-O-methyl (2′-OMe). Base modifications may include 5-position pyrimidine modifications, 8-position purine modifications, modifications at exocyclic amines, substitution of 4-thiouridine, substitution of 5-bromo- or 5-iodo-uracil, backbone modifications, methylations, unusual base-pairing combinations such as the isobases isocytidine and isoguanidine. Modifications can also include 3′ and 5′ modifications, such as capping. Other modifications can include substitution of one or more of the naturally occurring nucleotides with an analog, internucleotide modifications such as, for example, those with uncharged linkages (e.g., methyl phosphonates, phosphotriesters, phosphoamidates, carbamates, etc.) and those with charged linkages (e.g., phosphorothioates, phosphorodithioates, etc.), those with intercalators (e.g., acridine, psoralen, etc.), those containing chelators (e.g., metals, radioactive metals, boron, oxidative metals, etc.), those containing alkylators, and those with modified linkages (e.g., alpha anomeric nucleic acids, etc.). Further, any of the hydroxyl groups ordinarily present in a sugar may be replaced by a phosphonate group or a phosphate group; protected by standard protecting groups; or activated to prepare additional linkages to additional nucleotides or to a solid support. The 5′ and 3′ terminal OH groups can be phosphorylated or substituted with amines, organic capping group moieties of from about 1 to about 20 carbon atoms, or organic capping group moieties of from about 1 to about 20 polyethylene glycol (PEG) polymers or other hydrophilic or hydrophobic biological or synthetic polymers. Nucleic acids may be of variant types, such as locked nucleic acid (LNA), peptide nucleic acid (PNA), or gapmer.

A nucleic acid cargo may comprise DNA molecules.

The cargo may comprise an expression vector or expression cassette sequence. Suitable expression vectors and expression cassettes are known art. Expression vectors useful in the methods described herein comprise elements that facilitate the expression of one or more nucleic acid sequences in a target cell. Expression vectors useful in the present disclosure may comprise a transgene or other nucleic acid sequence.

An expression vector refers to an oligonucleotide molecule used as a vehicle to transfer foreign genetic material into a cell for expression in/by that cell. Such vectors may include a promoter sequence operably linked to the nucleotide sequence encoding the gene sequence to be expressed. A vector may also include a termination codon and expression enhancers. Any suitable promoters, enhancers and termination codons known in the art may be used.

In this specification the term “operably linked” may include the situation where a selected nucleotide sequence and regulatory nucleotide sequence (e.g. promoter and/or enhancer) are covalently linked in such a way as to place the expression of the nucleotide sequence under the influence or control of the regulatory sequence (thereby forming an expression cassette). Thus a regulatory sequence is operably linked to the selected nucleotide sequence if the regulatory sequence is capable of effecting transcription of the nucleotide sequence. Where appropriate, the resulting transcript may then be translated into a desired protein, peptide or polypeptide.

Examples of circular cargo molecules include minicircles and plasmids.

The nucleic acid cargo may be a minicircle. Minicircles are small (around 4 kbp) circular replicons. Minicircles usually comprise DNA, normally double stranded. Although minicircles occur naturally in some eukaryotic organelle genomes, minicircles preferred herein are synthetically derived. In some cases, the minicircle does not comprise an origin of replication, and thus does not replicate within the cell. Minicircles are known to those of ordinary skill in the art, e.g. see Gaspar et al., Minicircle DNA vectors for gene therapy: advances and applications. Expert Opin Biol Ther 2015 March; 15(3):353-79. doi: 10.1517/14712598.2015.996544. Epub 2014 Dec. 24. In some cases the minicircle comprises a reporter gene.

In some cases, the nucleic acid cargo is a plasmid. A plasmid is normally able to replicate independently in a cell. The plasmid may comprise an origin of replication sequence.

In some cases the nucleic acid is not modified to contain a sequence that binds to a protein on the surface of the vesicle. For example, the cargo nucleic acid does not contain a trans activating response (TAR) element. In some cases, the extracellular vesicle is not modified to contain a modified surface protein, such as an exogenous ARRDC1 protein or sequence derived from an ARRDC1 protein.

Extracellular vesicles according to the present disclosure may comprise (e.g. be loaded with) at least 0.1 nucleic acid molecules per vesicle. The extracellular vesicle(s) may comprise (e.g. be loaded with) one of 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0 or more copies of the nucleic acid per vesicle. The extracellular vesicle(s) may comprise (e.g. be loaded with) at least 0.5, at least 1, at least 2, at least 3, at least 3.5, at least 4, at least 5 or more copies of the nucleic acid per vesicle. The extracellular vesicle(s) may comprise (e.g. be loaded with) about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more copies of the nucleic acid per vesicle. The extracellular vesicle(s) may comprise (e.g. be loaded with) one of 0.1-1.0, 0.1-2.0, 0.1-3.0, 0.1-4.0, 0.1-5.0, 0.1-6.0, 0.1-7.0, 0.1-8.0, 0.1-9.0, 0.1-10, 0.1-15.0, 0.1-20.0, 0.1-25.0, 0.1-30.0, 0.1-35.0, 0.1-40.0, 0.1-45.0, 0.1-50, 1-2, 1-3, 1-4, 1-5, 1-6, 1-7, 1-8, 1-9, 1-10, 1-15, 1-20, 1-25, 1-30, 1-35, 1-40, 1-45, 1-50, 2-3, 2-4, 2-5, 2-6, 2-7, 2-8, 2-9, 2-10, 2-15, 2-20, 2-25, 2-30, 2-35, 2-40, 2-45, 2-50, 3-4, 3-5, 3-6, 3-7, 3-8, 3-9, 3-10, 3-15, 3-20, 3-25, 3-30, 3-35, 3-40, 3-45, 3-50, 4-5, 4-6, 4-7, 4-8, 4-9, 4-10, 4-15, 4-20, 4-25, 4-30, 4-35, 4-40, 4-45, 4-50, 5-6, 5-7, 5-8, 5-9, 5-10, 5-15, 5-20, 5-25, 5-30, 5-35, 5-40, 5-45, 5-50, 6-7, 6-8, 6-9, 6-10, 6-15, 6-20, 6-25, 6-30, 6-35, 6-40, 6-45, 6-50, 7-8, 7-9, 7-10, 7-15, 7-20, 7-25, 7-30, 7-35, 7-40, 7-45, 7-50, 8-9, 8-10, 8-15, 8-20, 8-25, 8-30, 8-35, 8-40, 8-45, 8-50, 9-10, 9-15, 9-20, 9-25, 9-30, 9-35, 9-40, 9-45, 9-50, 10-15, 10-20, 10-25, 10-30, 10-35, 10-40, 10-45, 10-50, 15-20, 15-25, 15-30, 15-35, 15-40, 15-45, 15-50, 20-25, 20-30, 20-35, 20-40, 20-45, 20-50, 25-30, 25-35, 25-40, 25-45, 25-50, 30-35, 30-40, 30-45, 30-50, 35-40, 35-45, 35-50, 40-45, 40-50, or 45-50 copies of the nucleic acid per vesicle.

The number of the nucleic acid(s) per vesicle may be an average number, preferably mean average, across a population of EVs, e.g. as present in a composition. The number of copies of nucleic acid per vesicle may be determined by dividing the total number of copies of the loaded nucleic acid cargo by the total number of EVs. In other words, Copies per EV=Number of loaded copies of nucleic acid/Total number of EV particles. The number of copies of nucleic acid may be determined by qPCR. The number of EVs may be determined by nanoparticle tracking analysis (NTA, e.g. as described in Wang et al., ARMMs as a versatile platform for intracellular delivery of macromolecules. Nature Communications 2018 9-960). Nanoparticle tracking analysis (NTA) is a method for visualizing and analyzing particles in liquids. The technique is used in conjunction with an ultramicroscope and a laser illumination unit that together allow small particles in liquid suspension to be visualized moving under Brownian motion. The light scattered by the particles is captured using a CCD or EMCCD camera over multiple frames. Computer software is then used to track the motion of each particle from frame to frame.

As used herein and unless indicated otherwise, the term “average” refers to the mathematical mean. This may refer to the total amount of nucleic acid determined in a sample, divided by the total number of vesicles in that sample

Although it may be desirable for the cargo to be loaded into substantially all of the extracellular vesicles in a composition, compositions disclosed herein may comprise extracellular vesicles in which one of at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, 98%, 99%, or 100% of the extracellular vesicles contain the cargo. Preferably, at least one of 50%, 55%, 60%, 65%, 70%, 75%, 80%, 85%, 90%, 95%, 96%, 97%, 98%, 99%, or 100% of the extracellular vesicles contain the cargo. In some cases, different extracellular vesicles within the composition contain different cargo. In some cases, the extracellular vesicles contain the same, or substantially the same, cargo molecule.

The size of a nucleic acid cargo may be defined in terms of its length in bases (for single stranded nucleic acids) or base pairs (for double stranded nucleic acids). In this specification, where the single or double stranded nature of the nucleic acid cargo is not indicated a length given in bases (e.g. in kb (kilobases) is also a disclosure of the same length in base pairs (e.g. in kbp). As such a length of 1 kb (1000 bases) is also a disclosure of 1 kbp (1000 base pairs). The term “bases” is used interchangeably with the term “nucleotides”. The nucleic acid cargo can be single stranded or double stranded. It can be linear or circular.

The nucleic acid cargo, e.g. RNA, may have a length of one of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 bases. The nucleic acid cargo may have a length of one of at least 50, 55, 60, 65, 70, 75, 80, 85, 90, 95 or 100 bases. The nucleic acid cargo may have a length of one of at least 100, 110, 120, 130, 140, 150, 160, 170, 180, 190, 200, 210, 220, 230, 240 or 250 bases.

Where the nucleic acid cargo is single stranded it may have a length of one of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 bases; at least 50, 55, 60, 65, 70, 75, 80, 85, 90, 95 or 100 bases; or at least 100, 110, 120, 130, 140, 150, 160, 170, 180, 190, 200, 210, 220, 230, 240 or 250 bases.

Where the nucleic acid cargo is single stranded it may have a length of one of at least 250, 500, 750, 1000, 1250, 1500, 1750, 2000, 2250, 2500, 2750, 3000, 3250, 3500, 3750, 4000, 4250, 4500, 4750, 5000, 5250, 5500, 5750, 6000, 6250, 6500, 6750, 7000, 7250, 7500, 7750, 8000, 8250, 8500, 8750, 9000, 9250, 9500, 9750, 10000, 10250, 10500, 10750 or 11000 bases. Optionally, wherein the nucleic acid cargo is single stranded it may have a maximum length of one of 4000, 4250, 4500, 4750, 5000, 5250, 5500, 5750, 6000, 6250, 6500, 6750, 7000, 7250, 7500, 7750, 8000, 8250, 8500, 8750, 9000, 9250, 9500, 9750, 10000, 10250, 10500, 10750 or 11000 bases. In preferred embodiments a single stranded nucleic acid cargo may have a minimum length of one of 2000, 2250, 2500, 2750, 3000, 3250, 3500, 3750, 4000, 4250, 4500, 4750, 5000 or more than 5000 bases.

Where the nucleic acid cargo is single stranded it may have a length of one of 250-750, 500-1000, 1000-1500, 1500-2000, 2000-2500, 2500-3000, 3000-3500, 3500-4000, 4000-4500, 4500-5000, 5000-5500, 5500-6000, 1000-2000, 2000-3000, 3000-4000, 4000-5000, 5000-6000, 6000-7000, 7000-8000, 8000-9000, 9000-10000, 10000-11000, 250-1000, 1000-3000, 1000-4000, 1000-5000, 1000-6000, 1000-7000, 1000-8000, 1000-9000, 1000-10000, 1000-11000, 2000-4000, 2000-5000, 2000-6000, 2000-7000, 2000-8000, 2000-9000, 2000-10000, 2000-11000, 3000-5000, 3000-6000, 3000-7000, 3000-8000, 3000-9000, 3000-10000, 3000-11000, 4000-6000, 4000-7000, 4000-8000, 4000-9000, 4000-10000, 4000-11000, 5000-7000, 5000-8000, 5000-9000, 5000-10000, 5000-11000, 6000-8000, 6000-9000, 6000-10000, 6000-11000, 7000-9000, 7000-10000, or 7000-11000, bases.

In some embodiments where the nucleic acid cargo is single stranded it may have a length of up to one of 2000, 3000, 4000, 5000, 6000, 7000, 8000, 9000, 10000, 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, or 40000 bases. The single stranded nucleic acid cargo may have a length of one of 5000-10000, 5000-15000, 5000-20000, 5000-25000, 5000-30000, 5000-35000, 5000-40000, 10000-15000, 10000-20000, 10000-25000, 10000-30000, 10000-35000, 10000-40000, 15000-20000, 15000-25000, 15000-30000, 15000-35000, 15000-40000, 20000-25000, 20000-30000, 20000-35000, 20000-40000, 25000-30000, 25000-35000, 25000-40000, 30000-35000, 30000-40000, or 35000-40000 bases.

Where the nucleic acid cargo is double stranded, e.g. double stranded RNA such as siRNA, it may have a length of one of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 base pairs. The nucleic acid cargo may have a length of one of at least 50, 55, 60, 65, 70, 75, 80, 85, 90, 95 or 100 base pairs. The nucleic acid cargo may have a length of one of at least 100, 110, 120, 130, 140, 150, 160, 170, 180, 190, 200, 210, 220, 230, 240 or 250 base pairs.

Suitable small molecules include cytotoxic reagents and kinase inhibitors. The small molecule may comprise a fluorescent probe and/or a metal. For example, the cargo may comprise a superparamagnetic particle such as an iron oxide particle. The cargo may be an ultra-small superparamagnetic iron oxide particle such as an iron oxide nanoparticle.

In some cases, the cargo is a detectable moiety such as a fluorescent dextran. The cargo may be radioactively labelled.

In some cases, the nucleic acid cargo are homogeneous (i.e. each nucleic acid in a composition of EVs is similar or substantially identical). In some cases, the nucleic acid cargo are heterogeneous (i.e. the nucleic acid in a composition of EVs are not similar or substantially identical to each other).

### Methods of Loading Extracellular Vesicles

In this specification, loading of an extracellular vesicle with a cargo refers to associating the extracellular vesicle and cargo in stable or semi-stable form such that the extracellular vesicle is useful as a carrier of the cargo, e.g. allowing its delivery to cells. Cargo molecules may be loaded in at least two ways. One is for the cargo to be present in the lumen of the extracellular vesicle (lumenal loading). Another is for the cargo to be attached to, adhered to, inserted through, or complexed with the external surface, e.g. membrane, of the extracellular vesicle (external surface loading). Cargo molecules loaded onto the external surface of the extracellular vesicle may usually be removed by contacting the vesicle with a nuclease, e.g. a DNase or RNase.

In some cases, extracellular vesicle(s), nucleic acid and transfection reagent are brought together under suitable conditions and for sufficient time to allow loading to occur.

Methods suitable for loading cargo into the extracellular vesicles are described in PCT/SG2018/050596 and include, for example, electroporation, sonication, ultrasound, lipofection or hypotonic dialysis.

Loading methods may include contacting a nucleic acid to be loaded with a transfection reagent, e.g. Exofect™ (System Biosciences).

Extracellular vesicles may be loaded by a combination of lumenal and external surface loading, and such extracellular vesicles may effectively deliver cargo nucleic acids to target cells.

Optionally, in some embodiments, reference to loading may be only to lumenal loading. Optionally, in some other embodiments, reference to loading may be only to external surface loading.

In some embodiments, loading of cargo into extracellular vesicles described herein does not comprise viral delivery methods, e.g. the loading methods do not involve a viral vector such as an adenoviral, adeno-associated, lentiviral, or retroviral vector.

### Immune Cells

The present invention relates to delivering nucleic acid to immune cells using RBCEVs. Also described are immune cells comprising RBCEVs and/or delivered nucleic acid, as well as immune cells expressing nucleic acid that has been delivered using RBCEVs.

The present invention includes immune cells comprising RBCEVs loaded with, or containing, a nucleic acid cargo.

It will be appreciated that where cells are referred to herein in the singular (i.e. “a/the cell”), pluralities/populations of such cells are also contemplated.

The immune cell according to the present disclosure may be a eukaryotic cell, e.g. a mammalian cell. The mammal may be a human, or a non-human mammal (e.g. rabbit, guinea pig, rat, mouse or other rodent (including any animal in the order Rodentia), cat, dog, pig, sheep, goat, cattle (including cows, e.g. dairy cows, or any animal in the order Bos), horse (including any animal in the order Equidae), donkey, and non-human primate.

In some embodiments, the cell may be from, or may have been obtained from, a human subject. Where the cell is to be administered to a subject, or where the cell is to be used in the preparation of a population of cells to be administered to a subject, the cell may be from a different subject to the subject to be administered (i.e. the cell may be allogeneic). In some cases the immune cell is an autologous cell (i.e. it is isolated, modified and then administered to the same subject from which it was isolated). In some cases the immune cell is an allogeneic cell (i.e. it is isolated, modified and then administered to a different subject from which it was isolated). Allogeneic cells are HLA matched to the recipient subject. In some cases the immune cell is a heterologous cell/a cell in a heterologous cell population (i.e. a cell is isolated, modified and then administered to a different subject from which it was isolated). Heterologous cells may be found in a mixed population of cells obtained from different donor subjects.

The immune cell may be a cell of hematopoietic origin, e.g. a neutrophil, eosinophil, basophil, dendritic cell, lymphocyte, or monocyte. The immune cell may be a mononuclear cell e.g. a peripheral blood mononuclear cell (PBMC). A lymphocyte may be e.g. a T cell, B cell, NK cell, NKT cell or innate lymphoid cell (ILC), or a precursor thereof. The immune cell may express e.g. CD3 polypeptides (e.g. CD3γ CD3ε CD3ζ or CD3δ), TCR polypeptides (TCRα, TCRβ, TCRγ, or TCRδ), CD27, CD28, CD4 or CD8.

In some embodiments the immune cell is a T cell, e.g. a CD3+ T cell. In some cases the T cell is a CD3+, CD4+ T cell. In some embodiments, the T cell is a CD3+, CD8+ T cell. In some embodiments, the T cell is a T helper cell (TH cell)). In some embodiments, the T cell is a cytotoxic T cell (e.g. a cytotoxic T lymphocyte (CTL)).

In some embodiments the immune cell is a primary cell. In some embodiments the immune cell is/has been obtained from a subject. In some embodiments the immune cell is/has been derived from an immune cell/population of immune cells obtained from a subject. In some embodiments the immune cell is not a cell of an immortalised cell line.

In some embodiments the immune cell is a naïve and/or undifferentiated T cell. The T cell may be a CD4+ T cell and express CD45RA, CCR7, CD62L and/or CD27. The T cell may be a CD8+, CD45RA+, CCR7+ T cell. in some cases the T cell does not express CD44, CD69, CD71, CD25 or HLA-DR. In some cases the T cell is not a CD45RA+CCR7− T cell.

In some embodiments the immune cell is a monocyte, e.g. a CD14+ monocyte. In some cases the monocyte expresses CD14, CD11 b, CCR2, and/or CD16.

In some embodiments the immune cell is a dendritic cell. hi some cases the dendritic cell expresses HLA-DR, CD80, CD86. The dendritic cell may be a conventional dendritic type 1 (cDC1) cell. The dendritic cell may be a conventional dendritic type 2 (cDC2) cell. The dendritic cell may be a plasmacytoid dendritic cell (PDC).

In some embodiments the immune cell is B cell, e.g. a CD19+ B cell. in some cases the B is an immature B cell. in some cases the B cell expresses CD20, CD34, CD38, and/or CD45R. in some cases the B cell does not express CD25 or CD30.

In some embodiments the immune cell is a macrophage. In some cases the macrophage is an alveolar macrophage. In some cases the macrophage is a tissue resident macrophage. In some cases the macrophage is a Kupffer cell. In some cases, the macrophage is a splenic macrophage.

In some embodiments the immune cell comprises/expresses an antigen receptor specific for an antigen of interest. In some embodiments the antigen receptor is a T cell receptor (TCR). In some embodiments the antigen receptor is a chimeric antigen receptor (CAR).

Methods of Delivering Nucleic Acid into Immune Cells

Methods disclosed herein involve a step of contacting an immune cell with an RBCEV for sufficient time, and under conditions suitable for the immune cell to take up the RBCEV. The RBCEV may be loaded with a nucleic acid cargo, as described herein.

In some aspects the present invention provides a method for delivering a nucleic acid into an immune cell, the method comprising incubating the immune cell with a RBCEV loaded with a nucleic acid cargo. It will be appreciated that where RBCEVs are referred to herein in the singular (i.e. “a/the RBCEV”), pluralities/populations of such RBCEVs are also contemplated.

Also provided is a method of transducing an immune cell with a nucleic acid comprising incubating the immune cell with a RBCEV loaded with a nucleic acid cargo.

The terms “incubating”/“incubation”/“incubate” are used herein to refer to placing the immune cell(s) and RBCEV(s) loaded with a cargo together at a suitable temperature and for a suitable time such that the RBCEV(s) are taken up, i.e. assimilated, incorporated or taken in, by the immune cell(s). These terms are also used herein to refer to bringing the immune cell(s) and loaded RBCEV(s) into sufficient contact that the immune cell(s) take up, i.e. assimilate, incorporate or take in, the RBCEV(s) and/or the cargo e.g. exogenous nucleic acid, e.g. during or after incubation. Incubation may produce the immune cell(s) described herein that comprise or contain at least one RBCEV and/or cargo. The immune cell(s) may be produced during and/or after incubation. Incubation may involve culturing the immune cells, or populations thereof, in vitro/ex vivo in cell culture medium comprising the cargo-loaded RBCEVs. Incubation may be performed as described in the Examples herein.

Incubation may be performed at a temperature close to body temperature of a mammal, e.g. at one or more of at least 35.0° C., at least 35.5° C., at least 36.0° C., at least 36.1° C., at least 36.2° C., at least 36.3° C., at least 36.4° C., at least 36.5° C., at least 36.6° C., at least 36.7° C., at least 36.8° C., at least 36.9° C., at least 37.0° C., at least 37.1° C., at least 37.2° C., at least 37.3° C., at least 37.4° C., and/or at least 37.5° C. In some cases the incubation is performed at two or more temperatures, e.g. as above. In some cases the incubation is performed at a single temperature. In some cases the incubation is performed at human body temperature. In some cases, incubation is performed at at least 37.0° C. In some cases, incubation is performed at 37.0° C. Incubation may be repeated on the same cells.

Incubation may comprise controlling the CO₂ level of the cell culture. Incubation comprising controlled CO₂ can control the pH of the incubated mixture. In some cases the CO₂ level of the incubating mixture is maintained at or close to the CO₂ level of blood, e.g. mammalian blood. In some cases incubation is performed at one or more of at least 4.0%, at least 4.1%, at least 4.2%, at least 4.3%, at least 4.4%, at least 4.5%, at least 4.6%, at least 4.7%, at least 4.8%, at least 4.9%, at least 5.0%, at least 5.1%, at least 5.2%, at least 5.3%, at least 5.4%, at least 5.5%, at least 5.6%, at least 5.7%, at least 5.8%, at least 5.9% and/or at least 6.0% CO₂. In some cases incubation is performed at one or more of at least 30 mmHg, at least 31 mmHg, at least 32 mmHg, at least 33 mmHg, at least 34 mmHg, at least 35 mmHg, at least 36 mmHg, at least 37 mmHg, at least 38 mmHg, at least 39 mmHg, at least 40 mmHg, at least 41 mmHg, at least 42 mmHg, at least 43 mmHg, at least 44 mmHg, and/or at least 45 mmHg CO₂.

In some cases incubation is performed at at least 5% CO₂. In some cases incubation is performed at about 5% CO₂. In some cases incubation is performed at 5% CO₂. In some cases incubation is performed at at least 38 mmHg CO₂. In some cases incubation is performed at about 38 mmHg CO₂. In some cases incubation is performed at 38 mmHg CO₂. In some cases incubation is performed in a humidified environment, e.g. in a humidified incubator.

Incubation may be performed for a length of time such that the RBCEVs are taken up by the immune cells. Incubation may be performed, e.g. at a combination of temperature and CO₂ level e.g. as above, for one of 12, 24, 36, 48, 60 or 72 hours. In some cases incubation is performed for at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, or at least 72 hours.

In some cases incubation is performed for at least 36 or at least 48 hours. In some cases incubation is performed for 48 hours. The methods described herein may comprise one or more steps of washing the immune cells after incubation, e.g. to remove any non-assimilated RBCEVs. Washing may be performed using PBS and centrifugation, e.g. at 4° C.

The methods described herein may comprise an incubation step comprising any combination of temperature, CO₂ level, and/or time, e.g. as described above. In some cases, incubation is performed at 37° C. at 5% CO₂ for 48 hours.

Incubation may be performed in any suitable medium, e.g. a cell culture medium. Suitable media for immune cells are well known in the art and described in e.g. Arora M, MATER METHODS 2013; 3:175.

In some cases incubation comprises shaking the mixture for some or all of the incubation time.

The method may include a step of loading the RBCEV with the nucleic acid cargo, e.g. as described herein. This step may be performed prior to incubating the RBCEV with the immune cell. This step may be performed separately to incubating the RBCEV with the immune cell.

In some cases the method does not include a step of loading the RBCEV with a nucleic acid cargo, e.g. the immune cells are incubated with a RBCEV that has been pre-loaded with a nucleic acid cargo.

In some cases the methods of delivering/transfecting an immune cell(s) with exogenous nucleic acid described herein do not comprise contacting the immune cell(s) with transfection reagents (although the RBCEVs themselves may be/have been loaded with nucleic acid cargo using e.g. transfection reagents).

In some cases, the method of delivering the nucleic acid into an immune cell is performed in vitro or ex vivo. In some cases, loading the RBCEV with the nucleic acid cargo is performed in vitro or ex vivo.

In some cases, the immune cell has been isolated from a subject, e.g. a human subject. The method may comprise an initial step of isolating an immune cell from a subject.

In some cases, the method comprises introducing or administering an immune cell comprising the nucleic acid into a subject, e.g. a human subject, e.g. as described herein. In some cases the methods described herein comprise reformulating the transfected immune cell(s), i.e. immune cell(s) comprising the nucleic acid, for administration to a subject. Reformulating may comprise mixing the immune cell(s) or population of immune cells with an adjuvant, diluent, or carrier suitable for administering to a mammal, e.g. a human.

In some embodiments, the methods described herein comprise testing whether the immune cell(s) comprising the RBCEV(s) and/or nucleic acid are activated and/or differentiated. In some cases, the methods comprise incubating the immune cell(s) and loaded RBCEV(s) as described herein, and then testing whether the immune cells are activated and/or differentiated. In some cases, immune cell(s) that are not activated and/or differentiated are isolated from immune cell(s) that are activated and/or differentiated. In some cases, immune cell(s) that are not activated and/or differentiated are used for subsequent applications, e.g. administering to a subject and/or for use in a method of treatment. In some cases, immune cell(s) that are activated and/or differentiated are not used for subsequent applications. Examples of methods for determining whether an immune cell is activated and/or differentiated are described herein below.

In some embodiments, the methods described herein comprise testing whether the immune cell(s) incubated with the RBCEV(s) loaded with nucleic acid express/are expressing the RBCEV-delivered nucleic acid. Suitable methods for determining if a cell expresses a nucleic acid are well known in the art and include e.g. qPCR for determining nucleic acid expression, and immunoassay based methods for detecting a translated protein, such as ELISA, flow cytometry, immunoblot, etc.

In some embodiments the method steps for production of an immune cell comprising a nucleic acid delivered by a RBCEV and/or comprising a RBCEV loaded with a nucleic acid may comprise one or more of: taking a blood sample from a subject; isolating immune cells, e.g. PBMCs or T cells, from the blood sample; generating/expanding a population of immune cells, e.g. T cells; culturing immune cells in in vitro or ex vivo cell culture; contacting the immune cells with a RBCEV loaded with a cargo, e.g. a nucleic acid; incubating the immune cells with a RBCEV loaded with a cargo, e.g. a nucleic acid; collecting immune cells comprising the nucleic acid; determining whether the immune cells express the nucleic acid; isolating the immune cells comprising/expressing the nucleic acid; determining whether the immune cells are activated/differentiated; isolating the immune cells that are not activated/differentiated; culturing the immune cells expressing the nucleic acid and/or that are not activated/differentiated in in vitro or ex vivo cell culture; mixing immune cells expressing the nucleic acid and/or that are not activated/differentiated with an adjuvant, diluent, or carrier; administering the modified immune cell to a subject.

Also provided is the use of a RBCEV loaded with a nucleic acid cargo for delivering the nucleic acid into an immune cell.

Also provided are immune cells and populations of immune cells obtained or obtainable by the methods described herein. Examples of the properties/characteristics of such cells, and how such cells may be identified and/or defined, are described below.

Also provided are methods comprising culturing an immune cell according to the present disclosure, e.g. in vitro/ex vivo, e.g. for generating/expanding a population of such cells. Methods for generating/expanding populations of immune cells in vitro/ex vivo are well known to the skilled person. Typical culture conditions (i.e. cell culture media, additives, temperature, gaseous atmosphere), cell numbers, culture periods, etc. can be determined by reference e.g. to Ngo et al., J Immunother. (2014) 37(4):193-203, which is hereby incorporated by reference in its entirety.

Conveniently, cultures of immune cells according to the present disclosure may be maintained at 37° C. in a humidified atmosphere containing 5% CO₂. The cells of cell cultures according to the present disclosure can be established and/or maintained at any suitable density, as can readily be determined by the skilled person. For example, cultures may be established at an initial density of ˜0.5×10⁶ to ˜5×10⁶ cells/ml of the culture (e.g. ˜1×10⁶ cells/ml).

Cultures can be performed in any vessel suitable for the volume of the culture, e.g. in wells of a cell culture plate, cell culture flasks, a bioreactor, etc. In some embodiments cells are cultured in a bioreactor, e.g. a bioreactor described in Somerville and Dudley, Oncoimmunology (2012) 1(8):1435-1437, which is hereby incorporated by reference in its entirety. In some embodiments cells are cultured in a GRex cell culture vessel, e.g. a GRex flask or a GRex 100 bioreactor.

### Properties of Immune Cells Comprising RBCEV-Delivered Nucleic Acid

Immune cells comprising a RBCEV and/or transfected with/comprising/expressing a RBCEV-delivered, i.e. exogenous, nucleic acid according to the present disclosure may be characterised or defined by reference to one or more functional properties.

Immune cells produced by the methods according to the present invention may be modified, e.g. genetically modified. In some cases the immune cell comprises and/or expresses a nucleic acid that has been delivered by a RBCEV, for example an mRNA. The mRNA may be translated by the immune cell into a protein.

The delivered nucleic acid cargo may provide the immune cell with novel and/or improved properties. For example, in embodiments wherein the nucleic acid encodes an antigen-specific receptor, immune cells comprising/expressing the nucleic acid may display effector immune activity against cells expressing the antigen for the antigen-specific receptor. In some cases, the nucleic acid encodes a receptor. In some embodiments, the nucleic acid encodes a ligand for a receptor. Receptors and ligands may be soluble/secreted or cell-membrane bound.

In some cases, the delivered nucleic acid encodes an antigen-specific receptor. Antigen-specific receptors include e.g. antibodies/immunoglobulins (including B cell receptors (BCRs)), T cell receptors (TCRs) and chimeric antigen receptors (CARs). Antigen-specific receptors may be soluble/secreted or cell-membrane bound.

In some embodiments, the delivered nucleic acid encodes an antigen for an antigen-specific receptor. In some embodiments, the nucleic acid encodes an antigen associated with a disease or disorder (e.g. a cancer, an infectious disease or an autoimmune disease).

The delivered nucleic acid may encode a costimulatory receptor (e.g. 4-1BB, OX40, CD28, CD27, ICOS, CD30 or GITR) or an immune checkpoint protein (e.g. PD-1, CTLA-4, LAG-3, TIM-3, TIGIT, VISTA or BTLA). The nucleic acid may encode a ligand for a costimulatory receptor (e.g. 4-1BBL, OX40L, CD80, CD86, CD70, ICOSL, CD30L or GITRL) or a ligand for an immune checkpoint protein (e.g. PD-L1, PD-L2, CD80, CD86, MHC Class II, Gal-9, CD112, CD155 or VSIG3).

In some cases the delivered nucleic acid is or encodes a nucleic acid capable of increasing and/or decreasing one or more activities of an immune cell comprising/expressing the nucleic acid. In some cases the immune cell comprises a gene and/or protein whose expression is/has been modified by the delivered nucleic acid. The gene and/or protein may be a species endogenous to the immune cell, i.e. in the genome of the cell, or may be a species exogenous to the immune cell, i.e. that has been introduced into the cell. Modification of gene/protein expression includes upregulation or downregulation of expression at the transcriptional level, and/or upregulation or downregulation of expression of mRNA at the translational level. For example, miRNA and siRNA function in RNA silencing and post-transcriptional regulation of gene expression. Post-transcriptional regulation can result in downregulation of an endogenous RNA targeted by the delivered nucleic acid. It may also result in the upregulation of gene(s)/protein(s) that are usually inhibited by an endogenous target RNA whose expression is downregulated by a delivered nucleic acid.

The methods described herein may deliver one or more components of a gene editing system, e.g. as described above, to an immune cell. Thus, the immune cell may comprise genetic material that has been edited, e.g. such that it contains single stranded break(s), double stranded break(s), and/or insertion(s) or deletion(s) of nucleotides. The immune cell may comprise genetic material containing an exogenous DNA template introduced by the delivered nucleic acid. The immune cell may comprise protein(s) translated from genetic material that is/has been edited by the delivered nucleic acid, e.g. such that the immune cell expresses modified/edited protein(s).

In some embodiments an immune cell comprising RBCEV-delivered nucleic acid according to the present disclosure displays one or more of the following properties:
a) Expression of the RBCEV-delivered nucleic acid;
b) Expression of an exogenous nucleic acid, i.e. that is/has been delivered by a RBCEV;
c) Permanent or transient expression of the RBCEV-delivered nucleic acid;
d) Increased expression of the RBCEV-delivered nucleic acid, e.g. as compared to an immune cell transfected with the same nucleic acid using non-RBCEV delivery, e.g. electroporation or nucleofection;
e) Presence of the RBCEV-delivered nucleic acid;
f) Increased presence of the RBCEV-delivered nucleic acid, e.g. as compared to an immune cell transfected with the same nucleic acid using non-RBCEV delivery, e.g. electroporation or nucleofection;
g) A naïve, undifferentiated or non-activated status;
h) Expression of cell markers that indicate a naïve, undifferentiated or non-activated status;
i) Increased expression of cell markers that indicate a naïve, undifferentiated or non-activated status, e.g. as compared to the expression of cell markers on an immune cell transfected with nucleic acid using non-RBCEV delivery, e.g. electroporation or nucleofection;
j) Increased maintenance of cell markers that indicate a naïve, undifferentiated or non-activated status, e.g. as compared to the expression of cell markers on an immune cell transfected with nucleic acid using non-RBCEV delivery, e.g. electroporation or nucleofection;
k) Decreased expression of cell markers that indicate an activated and/or differentiated status, e.g. as compared to the expression of cell markers on an immune cell transfected with nucleic acid using non-RBCEV delivery, e.g. electroporation or nucleofection;
l) Expression of cell markers CD4, CD45RA, CCR7, CD62L and/or CD27;
m) Increased maintenance of expression of cell markers CD4, CD45RA, CCR7, CD62L and/or CD27 after transfection with RBCEV-loaded nucleic acid, e.g. as compared to the expression of these cell markers on an immune cell transfected with nucleic acid using non-RBCEV delivery, e.g. electroporation or nucleofection;
n) Expression of cell markers CD8, CD45RA. and CCR7;
o) Increased maintenance of expression of cell markers CD8, CD45RA, and CCR7 after transfection with RBCEV-loaded nucleic acid, e.g. as compared to the expression of these cell markers on an immune cell transfected with nucleic acid using non-RBCEV delivery, e.g. electroporation or nucleofection;
p) Expression of cell markers CD14, CD11b, CCR2, and/or CD16;
q) Increased maintenance of expression of cell markers CD14, CD11 b, CCR2, and/or CD16 after transfection with RBCEV-loaded nucleic acid, e.g. as compared to the expression of these cell markers on an immune cell transfected with nucleic acid using non-RBCEV delivery, e.g. electroporation or nucleofection;
r) Expression of cell markers CD19, CD20, CD34, CD38, and/or CD45R;
s) Increased maintenance of expression of cell markers CD19, CD20, CD34, CD38, and/or CD45R after transfection with RBCEV-loaded nucleic acid, e.g. as compared to the expression of these cell markers on an immune cell transfected with nucleic acid using non-RBCEV delivery, e.g. electroporation or nucleofection;
t) Decreased expression of cell markers CD44, CD69 and/or CD62L, e.g. as compared to the expression of these cell markers on an immune cell transfected with nucleic acid using non-RBCEV delivery, e.g. electroporation or nucleofection;

u) increased expression of a nucleic acid and/or protein that is not the nucleic acid cargo;

v) Decreased expression of a nucleic acid and/or protein that is not the nucleic acid cargo;

w) Maintenance of cell viability, e.g. as compared to an immune cell transfected with nucleic acid using electroporation or nucleofection;

x) Increased cell viability, e.g. as compared to an immune cell transfected with nucleic acid using electroporation or nucleofection;

y) Decreased risk of cell death, e.g. as compared to an immune cell transfected with nucleic acid using electroporation or nucleofection;

z) Presence of nucleic acid-loaded RBCEVs in the immune cells.
aa) Decreased exhaustion of immune cells e.g. using RBCEVs loaded with anti-PD1 siRNAs
bb) Increased activation of immune cells e.g. using RBCEVs loaded with miR-29 ASO
cc) Long-term expression of therapeutic gene e.g. Chimeric antigen receptor (CAR) or transgenic T cell receptor (TCR) e.g. using CRISPR-CAS9 knock-in
dd) Expression of antigens (e.g. cancer antigens) for vaccination.

Immune cell populations can be identified and characterised by detecting the presence and number of cell-specific proteins, e.g. the Cluster of Differentiation (CD) proteins, which can be used as lineage-specific markers. Such markers can be detected using techniques well known to a person skilled in the art, e.g. flow cytometry, mass cytometry (CyTOF), immunohistochemistry or immunocytochemistry. The process of T cell activation is well known to the skilled person and described in detail, for example, in Immunobiology, 5th Edn. Janeway C A Jr, Travers P, Walport M, et al. New York: Garland Science (2001), Chapter 8, which is incorporated by reference in its entirety.

Nucleic acid expression and activity can be detected and quantified using common techniques in the field such as quantitative PCR analysis of RNA levels and/or by immunoassay based methods for detecting the relevant protein, such as ELISA, flow cytometry, immunoblot, etc.

Cell viability can be assessed using assays that are well known in the art, e.g. as described in Cobb L, MATER METHODS 2013; 3:2799 and Posimo et al., J Vis Exp. 2014; (83): 50645.

Cell death can be measured using techniques such as those described in e.g. Cummings et al., Curr Protoc Pharmacol. 2004 Sep. 1; 0 12:10. Cytotoxicity can be investigated, for example, using any of the methods reviewed in Zaritskaya et al., Expert Rev Vaccines (2011), 9(6):601-616, hereby incorporated by reference in its entirety, e.g. by ⁵¹Cr release assay. Cytotoxicity can also be investigated by xCELLigence assay, e.g. as described in Example 2 herein.

The presence of RBCEVs, e.g. RBCEVs loaded with nucleic acid, can be determined using e.g. labelled RBCEVs with any suitable detection agent e.g. fluorescent dye as described herein. The presence of RBCEVs can also be determined by detecting the presence or activity of the delivered nucleic acid, as above.

### Compositions

The present disclosure provides compositions comprising a population or a plurality of immune cells, e.g. as described herein. In some cases, the composition comprises one or more or a plurality of immune cells comprising and/or expressing an exogenous nucleic acid. In some cases the composition comprises one or more or a plurality of immune cells comprising an exogenous nucleic acid delivered by a RBCEV. In some cases, the composition comprises one or more or a plurality of immune cells comprising a RBCEV(s) loaded with a nucleic acid cargo and/or comprising an exogenous nucleic acid. In some cases, the composition comprises one or more immune cells produced by the methods described herein and/or having the characteristics described herein, e.g. expressing the RBCEV-delivered nucleic acid.

In some cases a composition comprising a population or plurality of immune cells also comprises a plurality of extracellular vesicles. In some cases a population or plurality of immune cells themselves comprise a plurality of extracellular vesicles.

The compositions described herein may comprise or consist of an isolated population of immune cells as provided herein.

In some cases the population of cells produced by the methods described herein is enriched for immune cells comprising exogenous, i.e. RBCEV-delivered, nucleic acid as compared to the cell population prior to undergoing the delivery/transduction methods of the present invention (i.e. the immune cells comprising exogenous nucleic acid are present at an increased frequency in the population following the delivery/transduction methods). The composition may be, or may include, a pharmaceutical composition or medicament. The composition may comprise one or more immune cells and/or extracellular vesicles, and optionally a pharmaceutically acceptable carrier, diluent or excipient. Pharmaceutical compositions may be formulated for administration by a particular route of administration. For example, the pharmaceutical composition may be formulated for intravenous, intratumoral, intraperitoneal, intradermal, subcutaneous, intranasal or other administration route.

In some cases, the compositions described herein comprise of RBCEVs. The RBCEVs may be loaded with a cargo. The composition may be for use in delivering the cargo to an immune cell.

Compositions may comprise a buffer solution. Compositions may comprise a preservative compound. Compositions may comprise a pharmaceutically acceptable carrier.

The nucleic acid-containing compositions of the invention can be stored and administered in a sterile physiologically acceptable carrier, where the nucleic acid is dispersed in conjunction with any agents which aid in the introduction of the nucleic acid into cells.

Various sterile solutions may be used for administration of the composition, including water, PBS, ethanol, lipids, etc. In some cases, the concentration of the nucleic acid will be sufficient to provide a therapeutic dose, which will depend on the efficiency of transport into the cells.

Compositions may be provided in frozen or lyophilised form.

### Applications of Immune Cells Comprising RBCEV-Delivered Nucleic Acid

The RBCEVs and compositions described herein can be used to introduce a nucleic acid of interest into an immune cell, e.g. a T cell. The immune cells comprising the nucleic acid of interest find use in therapeutic and/or prophylactic methods.

A method for treating/preventing a disease/condition in a subject is provided, comprising administering to a subject an immune cell comprising the nucleic acid of interest obtained or prepared by the delivery method of the present invention. Also provided is an immune cell comprising the nucleic acid of interest obtained or prepared by the delivery method of the present invention for use in a method of medical treatment/prophylaxis. Also provided is an immune cell comprising the nucleic acid of interest obtained or prepared by the delivery method of the present invention for use in a method for treating/preventing a disease/condition. Also provided is the use of an immune cell comprising the nucleic acid of interest obtained or prepared by the delivery method of the present invention in the manufacture of a medicament for use in a method for treating/preventing a disease/condition. Compositions comprising said immune cells are also provided for said methods of treating/preventing a disease or condition.

In some embodiments there is provided a method for treating/preventing a disease/condition in a subject, comprising administering to a subject an immune cell comprising a RBCEV loaded with a nucleic acid cargo. Also provided is an immune cell comprising a RBCEV loaded with a nucleic acid cargo for use in a method of medical treatment/prophylaxis. Also provided is an immune cell comprising a RBCEV loaded with a nucleic acid cargo for use in a method for treating/preventing a disease/condition. Also provided is the use of an immune cell comprising a RBCEV loaded with a nucleic acid cargo in the manufacture of a medicament for use in a method for treating/preventing a disease/condition. Compositions comprising said immune cells are also provided for said methods of treating/preventing a disease or condition.

In some embodiments there is provided a method for treating/preventing a disease/condition in a subject, comprising administering to a subject an immune cell comprising a nucleic acid that has been delivered by a RBCEV. Also provided is an immune cell comprising a nucleic acid that has been delivered by a RBCEV for use in a method of medical treatment/prophylaxis. Also provided is an immune cell comprising a nucleic acid that has been delivered by a RBCEV for use in a method for treating/preventing a disease/condition. Also provided is the use of an immune cell comprising a nucleic acid that has been delivered by a RBCEV in the manufacture of a medicament for use in a method for treating/preventing a disease/condition. Compositions comprising said immune cells are also provided for said methods of treating/preventing a disease or condition.

Immune cells obtained by the methods disclosed herein may be used in immunotherapy.

In some cases, the present disclosure contemplates the use of the immune cells comprising the delivered nucleic acid of interest in methods to treat/prevent diseases/conditions by adoptive cell transfer (ACT).

Adoptive cell transfer generally refers to a process by which cells (e.g. immune cells) are obtained from a subject, typically by drawing a blood sample from which the cells are isolated. The cells are then typically modified and/or expanded, and then administered either to the same subject (in the case of adoptive transfer of autologous/autogeneic cells) or to a different subject (in the case of adoptive transfer of allogeneic cells). The treatment is typically aimed at providing a population of cells with certain desired characteristics to a subject, or increasing the frequency of such cells with such characteristics in that subject. In the present disclosure, adoptive transfer may be performed with the aim of introducing a cell or population of cells into a subject, and/or increasing the frequency of a cell or population of cells in a subject.

In some embodiments, the subject from which the immune cell is isolated is the subject administered with the modified immune cell (i.e., adoptive transfer is of autologous cells). In some embodiments, the subject from which the immune cell is isolated is a different subject to the subject to which the modified immune cell is administered (i.e., adoptive transfer is of allogeneic cells).

Adoptive transfer of immune cells is described, for example, in Kalos and June 2013, Immunity 39(1): 49-60, and Davis et al. 2015, Cancer J. 21(6): 486-491, both of which are hereby incorporated by reference in their entirety. The skilled person is able to determine appropriate reagents and procedures for adoptive transfer of cells according to the present disclosure, for example by reference to Dai et al., 2016 J Nat Cancer Inst 108(7): djv439, which is incorporated by reference in its entirety.

In some embodiments, the methods comprise:
(a) introducing nucleic acid of interest into an immune cell in accordance with the methods of the present disclosure, and
(b) administering the immune cell comprising the nucleic acid of interest to a subject.

In some embodiments, the methods comprise:
(a) isolating an immune cell;
(b) introducing nucleic acid of interest into the immune cell in accordance with the methods of the present disclosure, and
(c) administering the immune cell comprising the nucleic acid of interest to a subject.

In some embodiments, the methods comprise:
(a) isolating an immune cell from a subject;
(b) generating/expanding a population of immune cells;
(c) introducing nucleic acid of interest into an immune cell in accordance with the methods of the present disclosure, and
(d) administering the immune cell comprising the nucleic acid of interest to a subject.

In some embodiments, the subject from which the immune cells are isolated is the same subject to which cells are administered (i.e., adoptive transfer may be of autologous/autogeneic cells). In some embodiments, the subject from which the immune cells are isolated is a different subject to the subject to which cells are administered (i.e., adoptive transfer may be of allogeneic cells).

Preferably, the method is an in vitro method. Preferably, the immune cell is contacted with the RBCEV in vitro.

In some embodiments the methods may comprise one or more of:
taking a blood sample from a subject;
isolating immune cells (e.g. PBMCs) from the blood sample;
generating/expanding a population of immune cells (e.g. T cells, dendritic cells);
culturing the immune cells in in vitro or ex vivo cell culture;
contacting the immune cells with a RBCEV comprising a nucleic acid of interest (cargo);
incubating the immune cells with a RBCEV comprising a nucleic acid of interest (cargo);
collecting/isolating immune cells comprising the nucleic acid cargo;
determining whether the immune cells express the nucleic acid;
collecting/isolating the immune cells comprising/expressing the nucleic acid;
determining whether the immune cells are activated/differentiated;
collecting/isolating the immune cells that are not activated/differentiated;
culturing the immune cells comprising the RBCEV and/or nucleic acid of interest in in vitro or ex vivo cell culture;
culturing the immune cells expressing the nucleic acid and/or that are not activated/differentiated in in vitro or ex vivo cell culture;
mixing immune cells comprising/expressing the nucleic acid of interest and/or that are not activated/differentiated, with an adjuvant, diluent, or carrier;
administering immune cells comprising/expressing the nucleic acid of interest to a subject.

In some embodiments, the methods may additionally comprise treating the immune cell to induce/enhance expression of the delivered nucleic acid and/or to induce/enhance proliferation or survival of immune cells comprising the delivered nucleic acid. For example, the nucleic acid may comprise a control element for inducible upregulation of expression of the nucleic acid in response to treatment with a particular agent.

In some embodiments treatment is ex vivo or in vitro by administration of the RBCEV to cells in culture ex vivo or in vitro. In some embodiments treatment is in vivo by administration of the RBCEV to immune cells in vivo.

It will be appreciated that where RBCEVs are referred to herein in the singular (i.e. “a/the RBCEV”), pluralities/populations of such RBCEVs are also contemplated.

In some cases, the immune cells are useful in methods of treatment involving T cell therapies, such as those involving CART cells and T cell receptor therapies.

The methods may be effective to reduce the development/progression of a disease/condition, alleviation of the symptoms of a disease/condition or reduction in the pathology of a disease/condition. The methods may be effective to prevent progression of the disease/condition, e.g. to prevent worsening of, or to slow the rate of development of, the disease/condition. In some embodiments the methods may lead to an improvement in the disease/condition, e.g. a reduction in the symptoms of the disease/condition or reduction in some other correlate of the severity/activity of the disease/condition. In some embodiments the methods may prevent development of the disease/condition a later stage (e.g. a chronic stage or metastasis).

The immune cell and/or the extracellular vesicle may comprise a therapeutic cargo. The therapeutic cargo may be a non-endogenous substance for interacting with a target gene in a target cell.

In some embodiments, the methods and immune cells described herein are useful for treating a subject suffering from a disorder associated with a target gene, the method comprising the step of administering an effective amount of an immune cell comprising RBCEV-delivered nucleic acid to said subject. The target gene may be a gene expressed by an immune cell, e.g. a T cell. The nucleic acid may inhibit or enhance the expression of the target gene, or it may facilitate gene editing to silence the particular gene.

In some embodiments, methods of treatment described herein involve treatment of a disease in a subject by expression of a protein or peptide from a nucleic acid cargo. In some embodiments, methods of treatment described herein involve treatment of a disease in a subject by inhibiting expression of a protein or peptide through the activity of a nucleic acid cargo.

In some embodiments, the methods and immune cells described herein are useful for treating a subject suffering from a disorder associated with dysfunctional immune cells, e.g. dysfunctional T cells. In some embodiments, the methods and immune cells described herein are useful for enhancing, ameliorating and/or improving an immune response in a subject.

In some cases, the methods and immune cells disclosed herein are particularly useful for the treatment of a genetic disorder, inflammatory disease, cancer, autoimmune disorder, cardiovascular disease or a gastrointestinal disease. In some cases, the disorder is a genetic disorder selected from thalassemia, sickle cell anemia, or genetic metabolic disorder. In some cases, the extracellular vesicles or immune cells are useful for treating a disorder of the liver, bone marrow, lung, spleen, brain, pancreas, stomach or intestine.

In certain aspects, the immune cells are useful for the treatment of cancer. Immune cells disclosed herein may be useful for inhibiting the growth or proliferation of cancerous cells. The cancer may be a liquid or blood cancer, such as leukemia, lymphoma or myeloma. In other cases, the cancer is a solid cancer, such as breast cancer, lung cancer, liver cancer, colorectal cancer, nasopharyngeal cancer, kidney cancer or glioma. In some cases, the cancer is located in the liver, bone marrow, lung, spleen, brain, pancreas, stomach or intestine.

The nucleic acid may inhibit or enhance the expression of a target gene, or perform gene editing to silence a particular gene.

Immune cells and compositions described herein may be administered, or formulated for administration, by a number of routes, including but not limited to systemic, intratumoral, intraperitoneal, parenteral, intravenous, intra-arterial, intradermal, subcutaneous, intramuscular, oral and nasal. Preferably, the immune cells are administered by a route selected from intratumoral, intraperitoneal or intravenous. Preferably, the RBCEVs are administered by a route selected from oral, nasal, inhaled, systemic, intravenous, intraperitoneal, parenteral, intra-arterial, intradermal, subcutaneous or intramuscular. The medicaments and compositions may be formulated in fluid or solid form. Fluid formulations may be formulated for administration by injection to a selected region of the human or animal body. Alternatively, fluid formulations may be aerosolized for inhalation.

Administration is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams &

Immune cells described herein may be administered alone or in combination with other treatments, either simultaneously or sequentially dependent upon the condition to be treated.

Extracellular vesicles loaded with a cargo as described herein may be used to deliver that cargo to a target cell. In some cases, the method is an in vitro method. The target cell is an immune cell, such as a tissue resident immune cell. For example, the tissue resident immune cell may be tissue resident leukocyte such as a dendritic cell, monocyte, macrophage neutrophil, T cell or B cell.

### Subjects

The subject in accordance with aspects of the present disclosure may be any animal or human. The subject is preferably mammalian, more preferably human. The subject may be a non-human mammal, but is more preferably human. The subject may be male or female. The subject may be a patient. Therapeutic uses may be in humans or animals (veterinary use).

A subject may have been diagnosed with a disease or condition requiring treatment, may be suspected of having such a disease/condition, or may be at risk of developing/contracting such a disease/condition. In some embodiments a subject may be selected for treatment according to the methods of the present invention based on characterisation for certain markers of such disease/condition.

The invention includes the combination of the aspects and preferred features described except where such a combination is clearly impermissible or expressly avoided.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

Where a nucleic acid sequence is disclosed herein, the reverse complement thereof is also expressly contemplated.

Methods described herein may preferably performed in vitro. The term “in vitro” is intended to encompass procedures performed with cells in culture whereas the term “in vivo” is intended to encompass procedures with/on intact multi-cellular organisms.

Aspects and embodiments of the present invention will now be illustrated, by way of example, with reference to the accompanying figures. Further aspects and embodiments will be apparent to those skilled in the art. All documents mentioned in this text are incorporated herein by reference.

### EXAMPLES

### Example 1

### Materials and Methods

### Mouse T Cell Isolation

Spleens were harvested from BALB/c mice (Jackson Lab), homogenized and treated with collagenase IV to obtain dissociated cells. Erythrocytes were removed using ACK buffer (Thermo Fisher Scientific). Remaining cells were centrifuged at 350×g for 5 min at 4° C. and resuspended in FACS buffer (Phosphate-buffered saline (PBS) with 0.5% BSA and 1 mM EDTA). T cells were enriched using Pan T Cell Isolation Kit (Miltenyi Biotec, Bergisch Gladbach, Germany). The bulk isolated cells were cultured in RPMI with 10% Fetal Bovine Serum (FBS) and 1% Pen-Strep in a humidified incubator at 37° C. and 5% CO₂.

### Human Peripheral Blood Mononuclear Cells (PBMCs) Isolation

Whole blood samples were obtained from healthy donors with informed consent (Hong Kong Red Cross or New York Blood Center). Plasma and red blood cells were separated using centrifugation. Leukocyte-enriched blood from leukocyte filter chamber was backflushed and diluted in PBS with EDTA. Blood samples were layered on Ficoll-Paque PLUS (GE Healthcare Life Science). PBMCs were collected from the interface layer between plasma and ficoll after centrifugation at 400×g for 30 min, 4° C. The isolated cells were cultured in RPMI with 10% Fetal Bovine Serum (FBS) and 1% Pen-Strep in a humidified incubator at 37° C. and 5% CO₂ or freezed down in media with 10% DMSO and 90% FBS for further use.

### Human T Cell Isolation

Human CD4 and CD8 T cells were isolated from PBMCs by positive selection using anti-human CD4 and CD8 magnetic beads, respectively. For small RNA transfection, isolated CD8 T cells were cultured in RPMI with 10% Fetal Bovine Serum (FBS), 2 ng/mL IL-7, and 1% Pen-Strep in a humidified incubator at 37° C. and 5% CO₂. For RNA transfection, CD4 and CD8 T cells were cultured in RPMI with 10% FBS, 200 U/mL IL-2, 5 ng/mL IL-7, 5 ng/mL IL-15, 1% Pen-Strep and 1× plasmocin.

### Generation of Monocytes-Derived Dendritic Cells

PBMCs were resuspended in RPMI 1640 supplemented with 10% FBS and 0.1% penicillin-streptomycin. After 6 hours, the adherent cells (monocytes) were washed twice with RPMI 1640 and then incubated with complete RPMI 1640 medium, supplemented with 1000 U/ml human GM-CSF and 500 U/ml human IL-4. After 5 days, the medium was replaced with complete RPMI 1640 containing 50 ng/ml human TNFα and 10 ng/ml human IL-1β. On Day 7, loosely adherent DCs were harvested and analysed for HLA-DR, CD80 and CD86 expression using FACS.

### RBCEV Purification

RBCEVs were purified according to our previous study (Waqas et al., 2018) with minor changes. Briefly, leukocytes and plasma were completely removed. RBCs were diluted in RBC buffer (1:1). 1 μL of 10 mM calcium ionophore was added into 1 mL of RBC solution and the cells were incubated at 37° C. overnight. The cells were pelleted and supernatant was collected by centrifugation at increasing speed (600×g for 20 min, 1,600×g for 15 min, and 3,260×g for 15 min). The supernatant was filtered through a 0.45 μm membrane before a centrifugation at 100,000×g for 70 min at 4° C. using a SW32 rotor. The pellet was resuspended in 1 mL of PBS and subsequently loaded onto a 60% sucrose cushion and ultracentrifuged at 100,000×g for 16 h at 4° C. in an SW32 rotor. RBCEVs were collected at the interface and diluted in PBS for a final wash at 100,000×g for 70 min. RBCEVs were diluted in PBS. Each batch of RBCEVs was sampled for particle concentration (Nanosight), protein concentration (BCA assay), and haemoglobin concentration.

### RBCEV Labelling and Uptake Assay

1 mg of RBCEVs were incubated with 20 μM CFSE fluorescent dye (Thermo Fisher Scientific) for 1 h at 37° C. Excessive dye was removed by centrifugation at 21,000×g for 30 min at 4° C. RBCEV pellet was resuspended in PBS and loaded into a qEV original Izon column (Izon Science). RBCEV fractions were collected and centrifuged at 21,000×g. RBCEVs pellet was resuspended in PBS and quantified using a haemoglobin assay kit (Abcam).

100 μg of CFSE labelled RBCEVs were incubated with 300,000 cells mouse spleen T cells or human PBMCs or with 500,000 dendritic cells in 500 μL of medium at 37° C., 5% CO₂ in a humidified incubator. After 24 or 48h, cells were collected and washed twice with PBS by centrifugation at 350×g for 5 min at 4° C. The cell pellet was resuspended in 200 μI of FACS buffer. Treated cells were then subjected to flow cytometry analysis to detect CFSE signals.

Loading RNA or DNA into RBCEVs

Antisense oligonucleotides (ASOs) or miRNA mimics (Thermo Fisher) were loaded into RBCEVs using transfection reagents or electroporation. For electroporation, 75 μg RBCEVs were mixed with 400 pmol ASOs or miRNA mimics in 100 μL OptiMEM (Thermo) on ice for 10 min. The solution was loaded into an electroporation cuvette (Biorad) and electroporated using the Gene Pulser Xcell system (Biorad) at 250V, 100 μF, and exponential current. Loaded EVs were then incubated with 300,000 CD8 T cells for 24h before new medium was added. For transfection, 1 μg ASO (Shanghai GenesPharma), mRNA (Trilink) or GFP-MC plasmid (Carmine) was mixed with 2.5 μl transfection reagent and incubated at room temperature for 20 minutes then incubated with 50 μg RBCEVs at room temperature for 30 minutes with shaking. Afterwards, free RNA and transfection reagents were washed away using size exclusion chromatography or centrifugation. 100 μg of RNA/DNA loaded EVs were incubated with 300,000 cells. After 48h, cells were washed and analyzed by flow cytometry.

As controls, 300,000 CD8 T cells were nucleofected or electroporated with 400 pmol FAM-labelled NC-ASO using an Amaxa T cell nucleofection kit (Lonza) or a Neon transfection system (Thermo), respectively, following the manufacturer protocols. Briefly, for nucleofection, 900,000 cells were mixed with 100 μl human T cell nucleofector solution and 1,200 pmol FAM-NC-ASO. The cell solution was then loaded into a cuvette and nucleofected using program U-014 for high viability. After nucleofection, cells were resuspended into preconditioned medium at 37° C. For electroporation, 900,000 cells were resuspended in 100 μl of buffer T with 1,200 pmol FAM-NC-ASO. Cells were loaded onto a 100 μl-Neon tip and electroporated. After electroporation, cells were resuspended in preconditioned complete medium without antibiotics. Cells were collected for flow cytometry analysis at 24 and 120h. 72h after 100,000 CD8 T cells were incubated with ASO/miRNA-loaded RBCEVs, treated cells were also collected for RNA extraction and qRT-PCR. After 120h, the RBCEV-treated cells were stained with anti-EOMES and anti-TBET antibodies for flow cytometry analysis.

Delivery of mRNA and Plasmid DNA to Mouse Splenocytes Using RBCEVs

Splenocytes were dissociated from spleens of C57BL/6 mice using collagenase IV at 37° C. for 30 minutes. RBCEVs were loaded with mCherry mRNA or GFP-MC plasmid and washed as described above. 0.5 mg of loaded RBCEVs were incubated with splenocytes for 48 hours. Splenocytes were washed twice with PBS and incubated with fluorescent antibodies that recognize CD11c, CD11b, CD103, NK1.1, CD8 and CD4 (Biolegends) for FACS analysis.

### Results

Mouse T Cells Readily Take Up Human-Derived RBCEVs without Activation

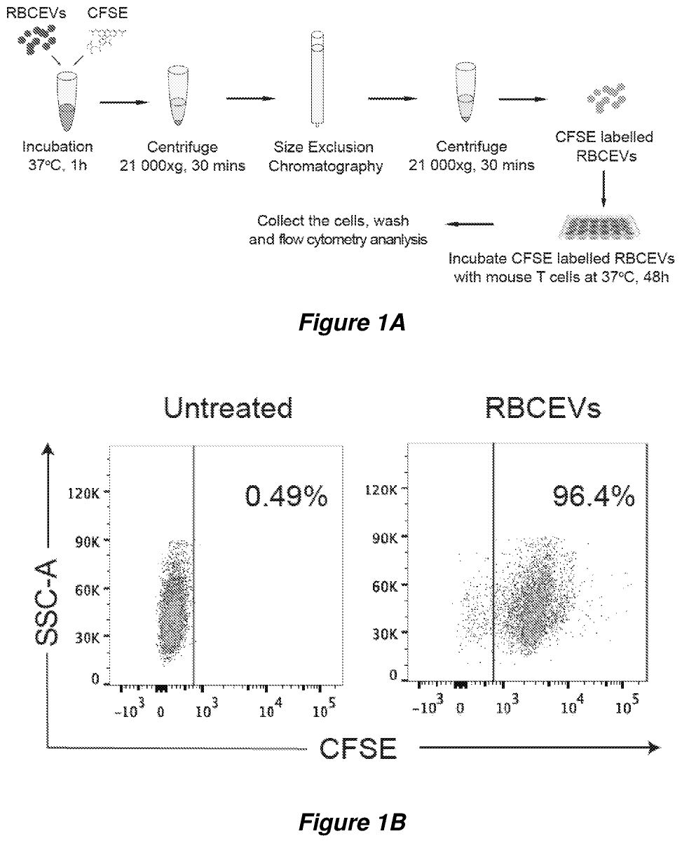

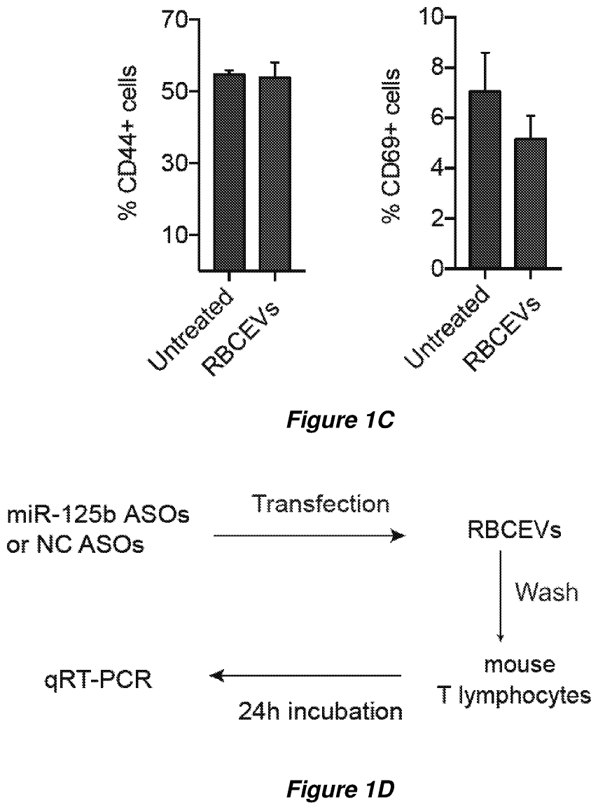

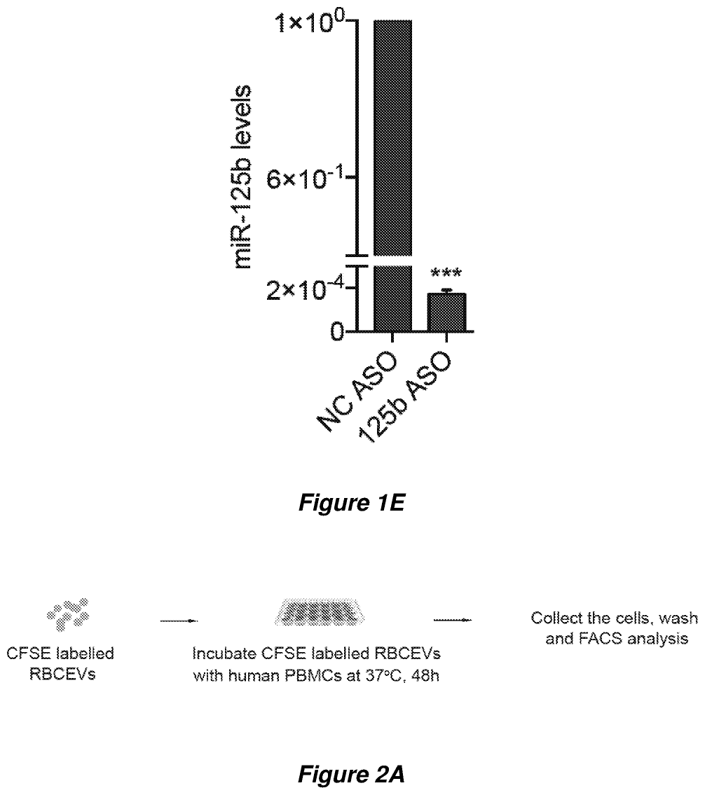

To explore the potential of RBCEVs to deliver nucleic acids to T cells, CD3+ T cells were isolated from mouse spleen and incubated with CFSE-labelled RBCEVs. CFSE in the cells was analysed using FACS (FIG. 1A). After 48h, all the cells became CFSE positive (FIG. 1B). T cell activation markers, CD44 and CD69, in RBCEV-treated mouse T cells were examined. Compared to untreated samples, the percentage of CD44+ and CD69+ cells remained unchanged after RBCEV treatments (FIG. 1C). To verify the ability of RBCEVs to deliver functional cargos, miR-125b antisense oligonucleotides (ASOs) were loaded into RBCEVs then the loaded RBCEVs were incubated with mouse CD3+ T cells (FIG. 1D). After 24 hours, the levels of miR-125b reduced significantly in CD3+ T cells treated with miR-125b ASOs loaded RBCEVs (FIG. 1E). Hence, RBCEVs are readily taken up by mouse T cells without activation and the cargoes in RBCEVs are functional after the uptake.

### RBCEVs Deliver RNA ASOs to Human PBMCs Including T Cells

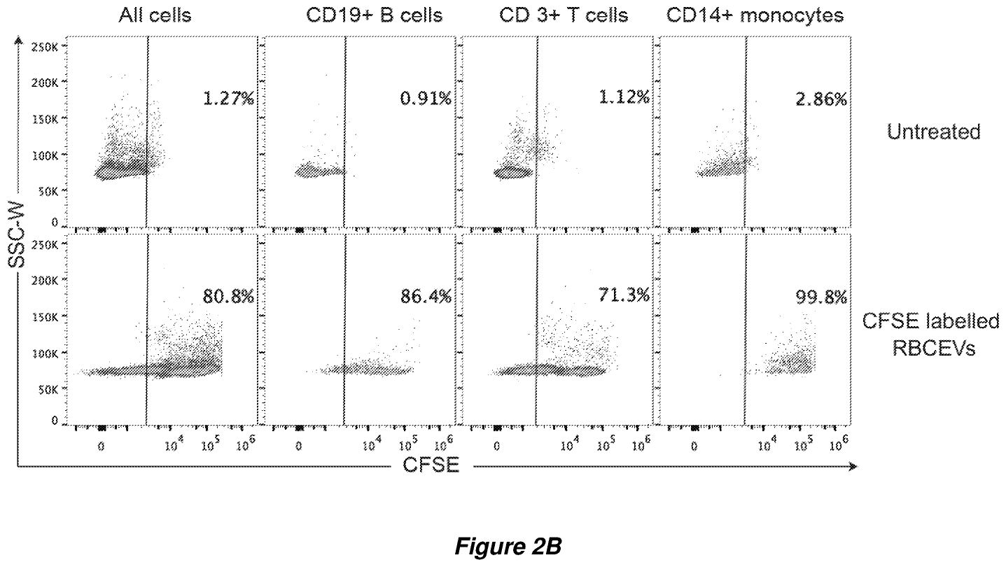

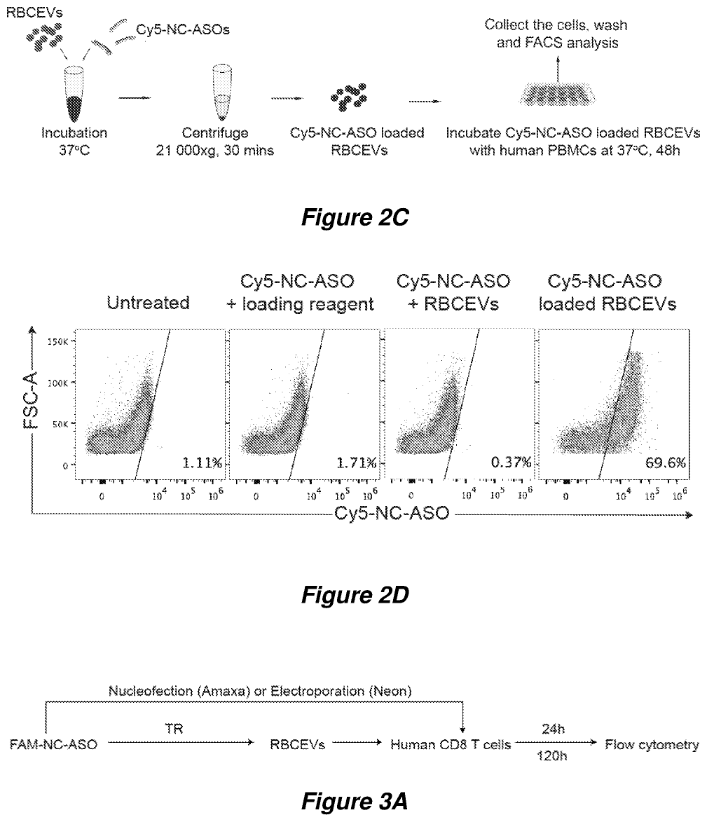

Human PBMCs were incubated with CFSE-labelled RBCEVs to test the uptake of RBCEVs (FIG. 2A). After 48h, about 80% of all PBMCs became CFSE positive, analyzed by FACS (FIG. 2B). Among subpopulations of PBMCs, CD14+ monocytes were the most robust recipients of RBCEVs with about 99% of the cells became CFSE positive compared to untreated control (FIG. 2B). CD19+ B cells and CD3+ T cells also took up RBCEVs at the rate of 86% and 71%, respectively (FIG. 2B).

Further, RBCEVs were loaded with a Cy5 labelled non-targeting ASO (Cy5-NC-ASO) and then incubated with human PBMCs (FIG. 2C). After 48h, about 70% of PBMCs became Cy5 positive (FIG. 2D). Incubation of the cells with Cy5-NC-ASO and loading reagent only or RBCEVs with unloaded Cy5-NC-ASO did not increase the Cy5 signals in human PBMCs compared to non-treated samples (FIG. 2D). These results indicate that RBCEVs can effectively deliver ASOs to human PBMCs including the T cell populations.

RBCEVs Deliver RNAs to Human CD8 T Cells More Effectively than Other Transfection Methods

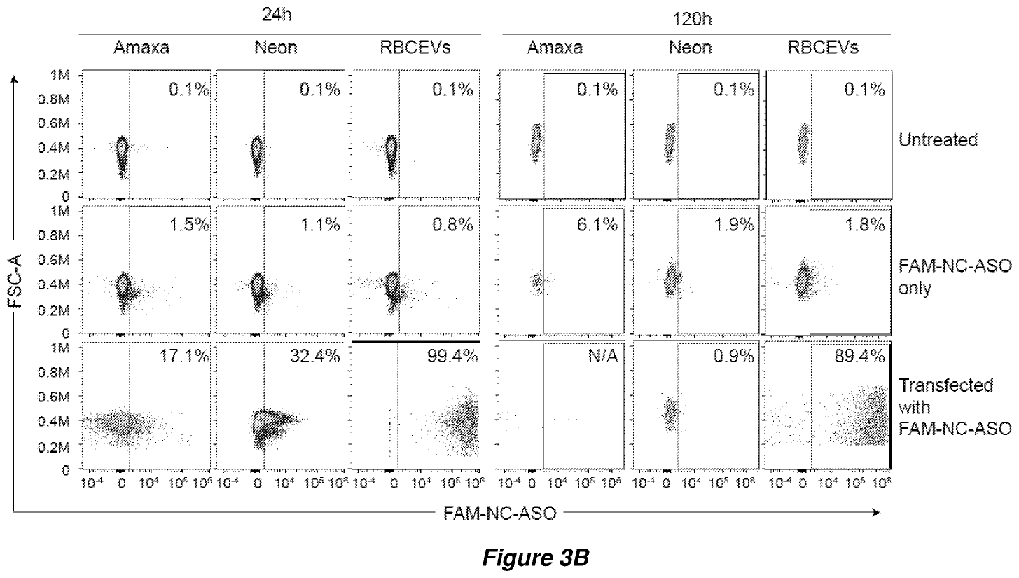

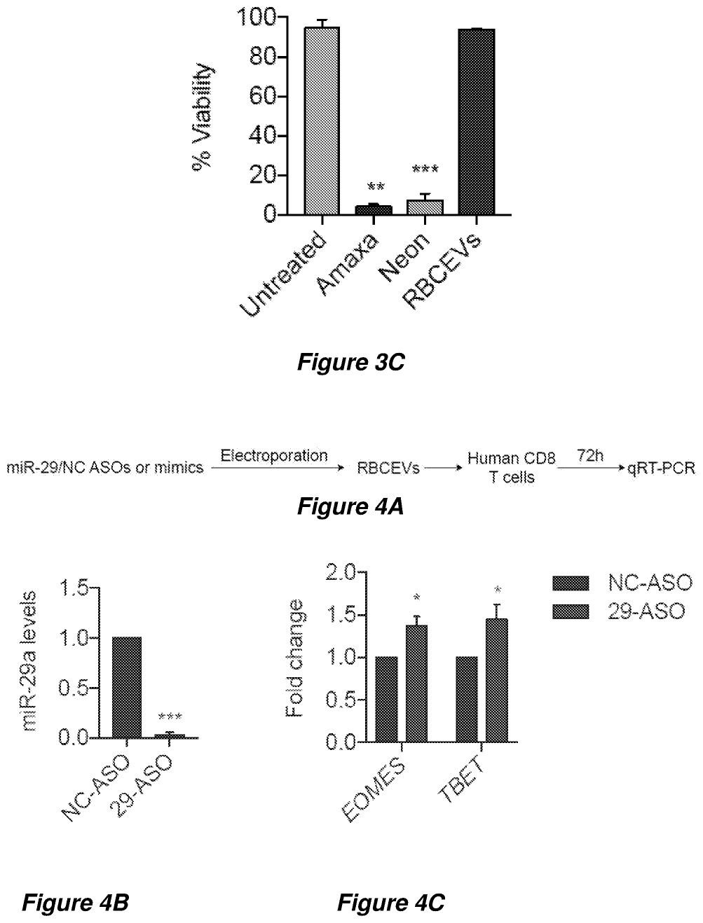

To compare the efficiency of RNA delivery by RBCEVs with common transfection methods for lymphocytes, FAM-labelled ASOs were delivered to human CD8 T cells using RBCEVs, cell electroporation, or nucleofection (FIG. 3A). At 24h post-transfection, 99.4% of cells treated with FAM-NC-ASO loaded RBCEVs were highly positive for FAM signal as compared to electroporation and nucleofection of T cells using commercial Neon kit (Thermo) and Amaxa kit (Lonza) respectively (FIG. 3B). Moreover, not only did the other transfection methods yield lower FAM signals (17-32% FAM+) in the cells at 24h post-transfection but these signals diminished gradually after 5 days. Most of the nucleofected cells and part of the electroporated cells died as a result of these treatments. Meanwhile, CD8 T cells treated with RBCEVs survived well and maintained the FAM signal (89.4%) up to 5 days (FIG. 3C). Therefore, RBCEVs are more effective than nucleofection and electroporation methods in delivering RNAs into human lymphocytes for a prolonged period of time.

Delivery of Functional ASOs and miRNA Mimics into CD8 T Cells by RBCEVs

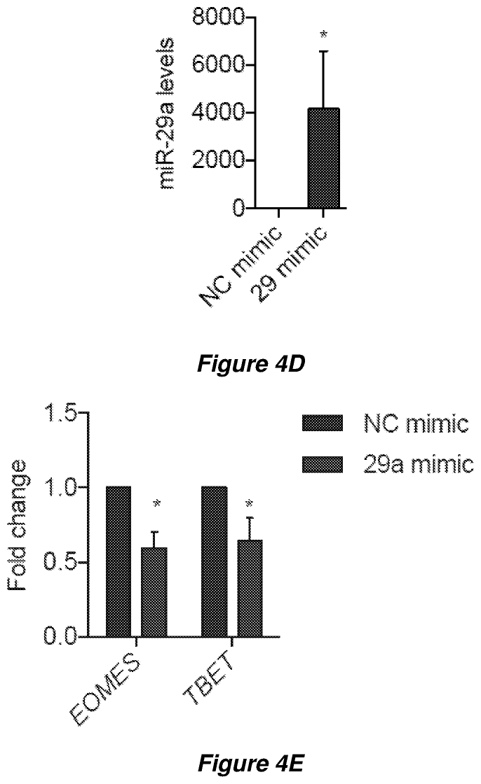

miR-29a is an important regulator of effector T cell functions. It is abundant in adult CD8 T cells but low in neonatal CD8 T cells.⁷ Experiments were performed to suppress miR-29a in adult CD8 T cells or overexpress this miRNA in cord-blood-derived CD8 T cells using miR-29a ASO or miR-29a mimics in RBCEVs, respectively (FIG. 4A). After 72h, cellular miR-29a were significantly downregulated in adult CD8 T cells treated with RBCEVs containing miR-29 ASOs while miR-29a's targets, EOMES and TBET were upregulated (FIG. 4B-C). On the other hand, miR-29a levels in cord-blood CD8 T cells increased by ˜4000 times after treatment with RBCEVs loaded with miR-29a mimics compared to NC mimic treated controls (FIG. 4D). We also observed a significant downregulation of EOMES and TBET as the consequence of miR-29a overexpression (FIG. 4E).

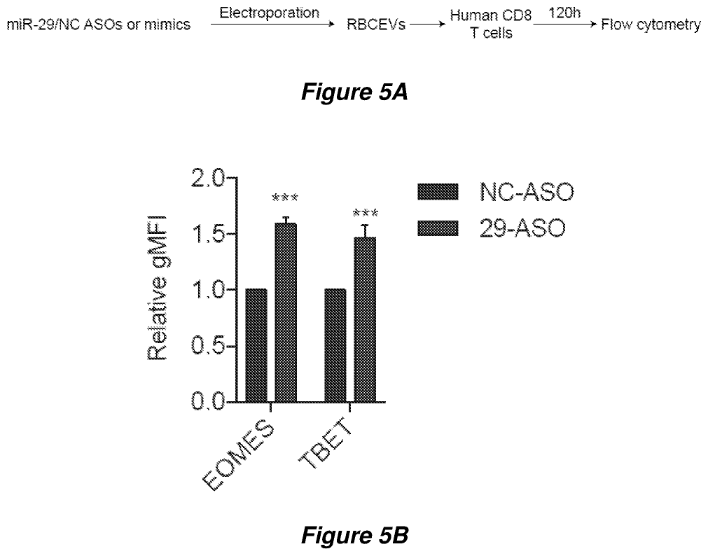

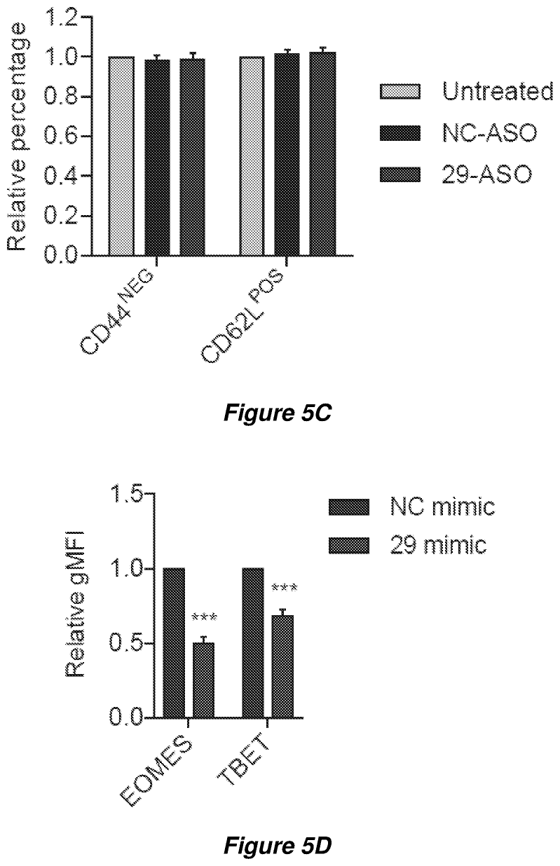

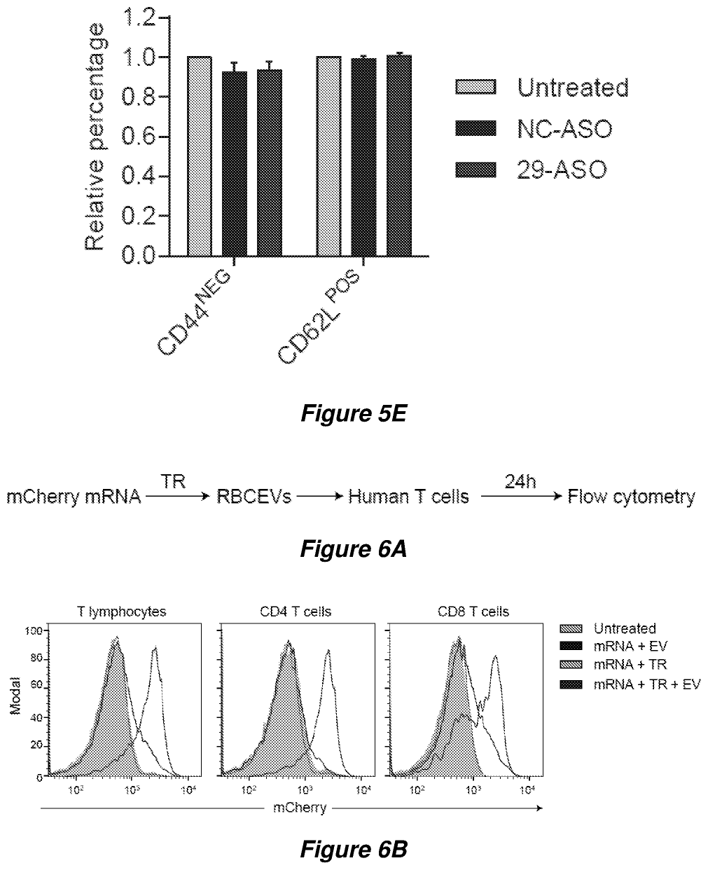

Consistently, analysis by flow cytometry showed that after 120h of treatment with miR-29a ASO or mimic-loaded RBCEVs, cellular levels of EOMES and TBET increased in adult CD8 T cells and decreased in cord blood CD8 T cells, respectively (FIG. 5B, D). Common transfection strategies often lead to activation and differentiation of naïve CD8 T cells. However, we found that both adult and cord blood naïve CD8 T cells remained inactive and undifferentiated after RBCEV treatments (FIG. 5C, E). Therefore, RBCEVs can effectively transfer functional RNAs to human naïve lymphocytes without activating or differentiating the cells.

Delivery of Functional mRNAs into Lymphocytes by RBCEVs

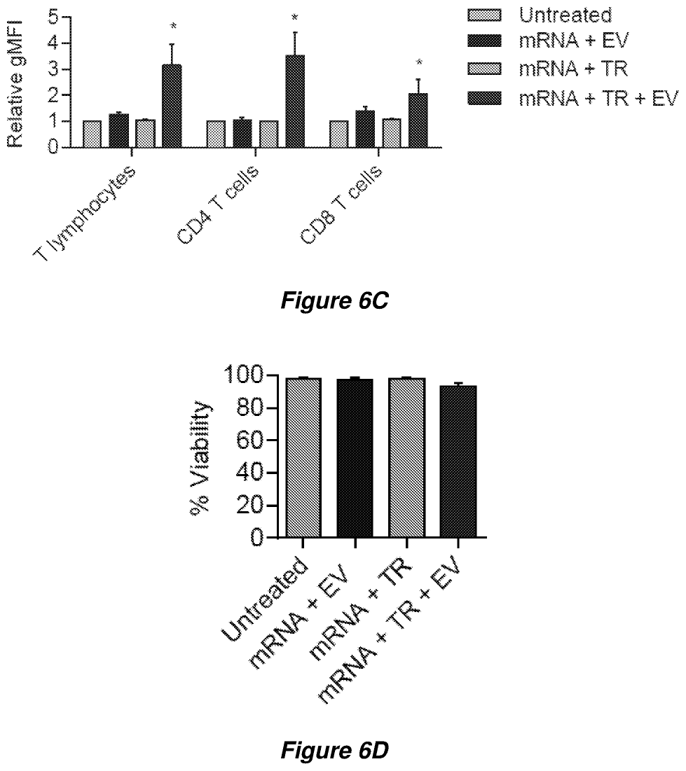

mCherry mRNAs were loaded into RBCEVs and the loaded RBCEVs were added into isolated human lymphocytes (FIG. 6A). After 24h, mCherry signals were detected from cells treated with RNA-loaded RBCEVs in both CD4 and CD8 T cells (FIG. 6B-C). Moreover, cells retained high viability after the delivery (FIG. 6D). Therefore, RBCEVs are capable of delivering mRNAs to lymphocytes.

Delivery of Functional Plasmid into Human Lymphocytes by RBCEVs

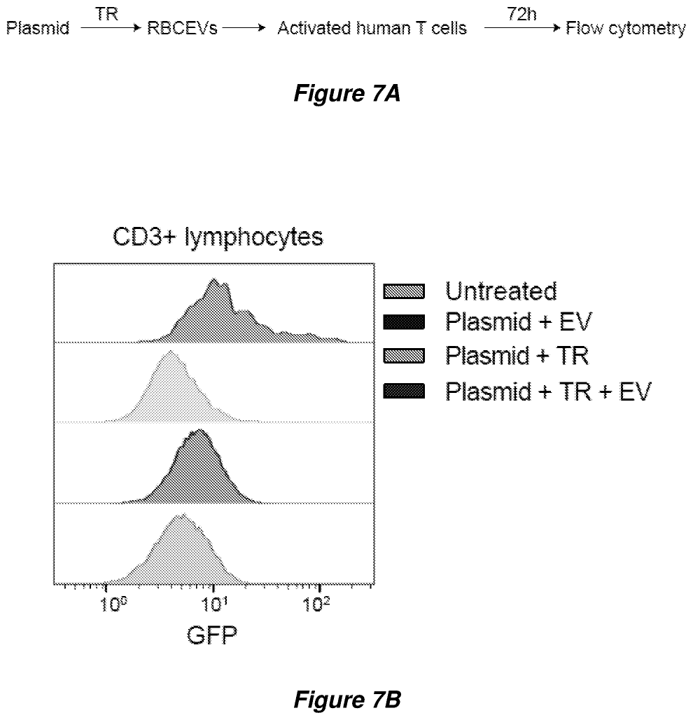

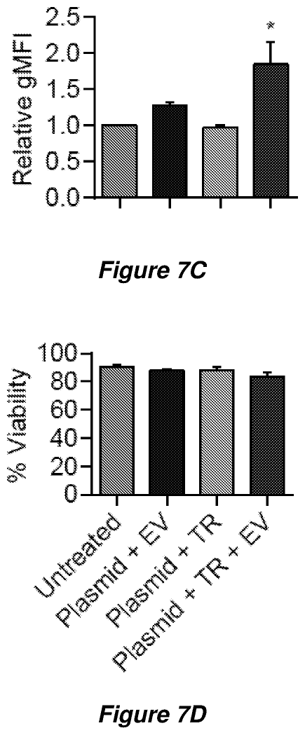

Minicircle plasmid containing GFP sequence (MC-GFP) were loaded into RBCEVs and added the mixture into activated human lymphocytes (FIG. 7A). After 72h, GFP signal was detected from CD3 positive cells treated with plasmid loaded RBCEVs (FIG. 7B-C). Moreover, cells retained high viability after the delivery (FIG. 7D). In summary, RBCEVs are very efficient in delivering different types of nucleic acids into lymphocytes.

Delivery of Cas9 mRNA into Human Lymphocytes Using RBCEVs

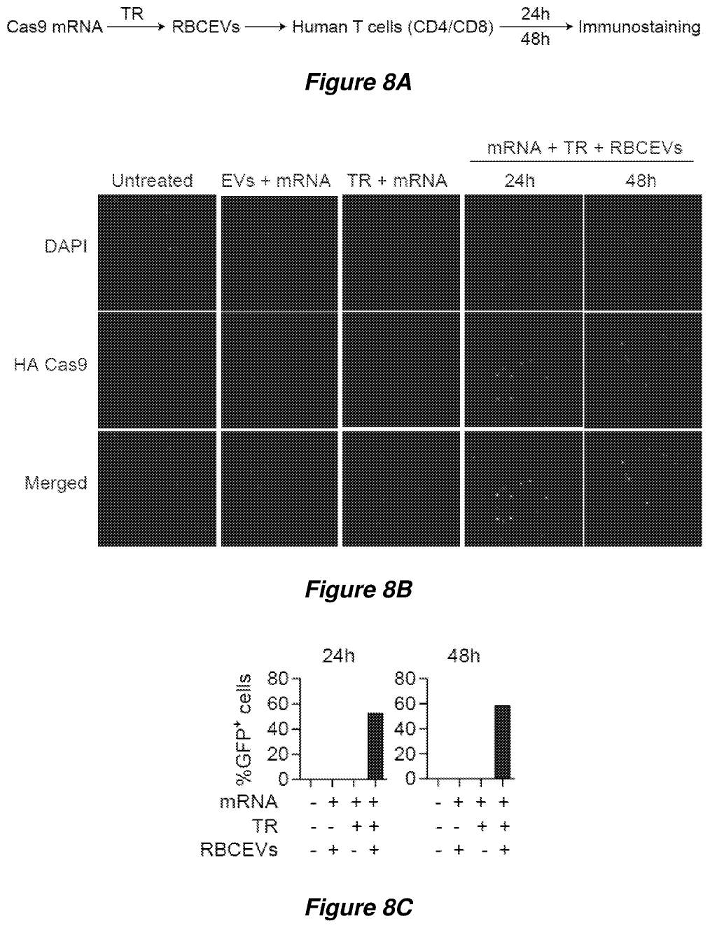

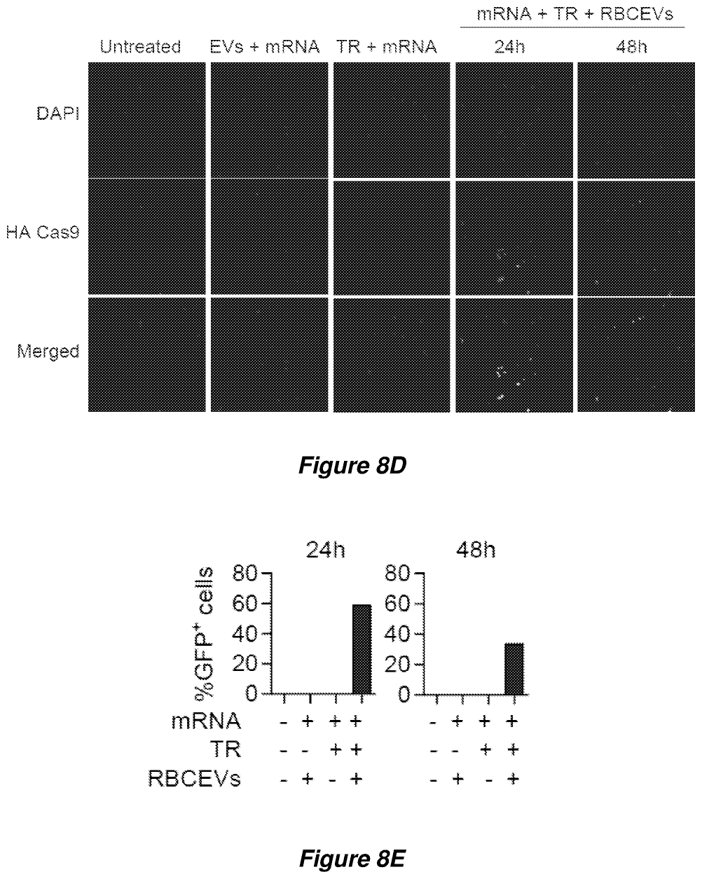

To test the feasibility of CRISPR-Cas9 delivery in T cells, we loaded RBCEVs with an HA-tagged Cas9 mRNA and incubated them with CD4+ and CD8+ human T cells for 24 to 48 hours (FIG. 8A). Using immunostaining of HA tag, we found that Cas9-HA protein was expressed in the nuclei of ˜50% CD4+ T cells after 24-48 hours (FIG. 8B-C). The same was observed in CD8+ T cells but Cas9-HA was found initially in ˜60% of the cells at 24 hours and only ˜38% of the cells at 48 hours (FIG. 8D-E). This data suggests that RBCEVs delivered Cas9 mRNA that was translated into Cas9 protein in the nucleus. Hence, Cas9 mRNA can be used for CRISPR-Cas9 genome editing in T cells.

### RBCEVs are Taken Up by Human Dendritic Cells

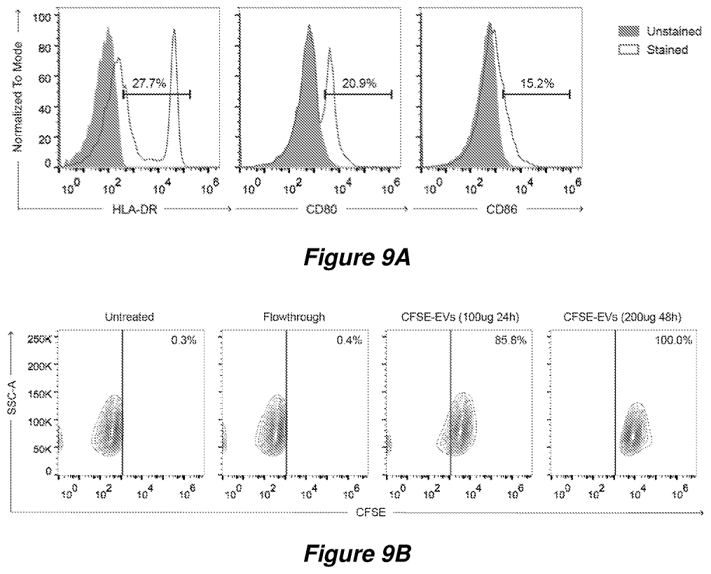

To test if RBCEVs are taken up by dendritic cells, we adhered human PBMCs to cell culture plates and differentiate them into mature dendritic cells using a cytokine cocktail for 1 week. FACS analysis revealed the expression of mature dendritic cell markers including HLA-DR, CD80 and CD86 in 15-28% of the cells (FIG. 9A). Incubation with CFSE-labelled RBCEVs led to 86% uptake after 24 hours and 100% uptake after 48 hours, based on the percentage of CFSE-positive cells (FIG. 9B). This data suggests that dendritic cells take up RBCEVs readily hence RBCEVs can be used to deliver therapeutic molecules to dendritic cells for immunotherapies.

Delivery of mRNA and Plasmid DNA to Mouse Splenocytes Using RBCEVs

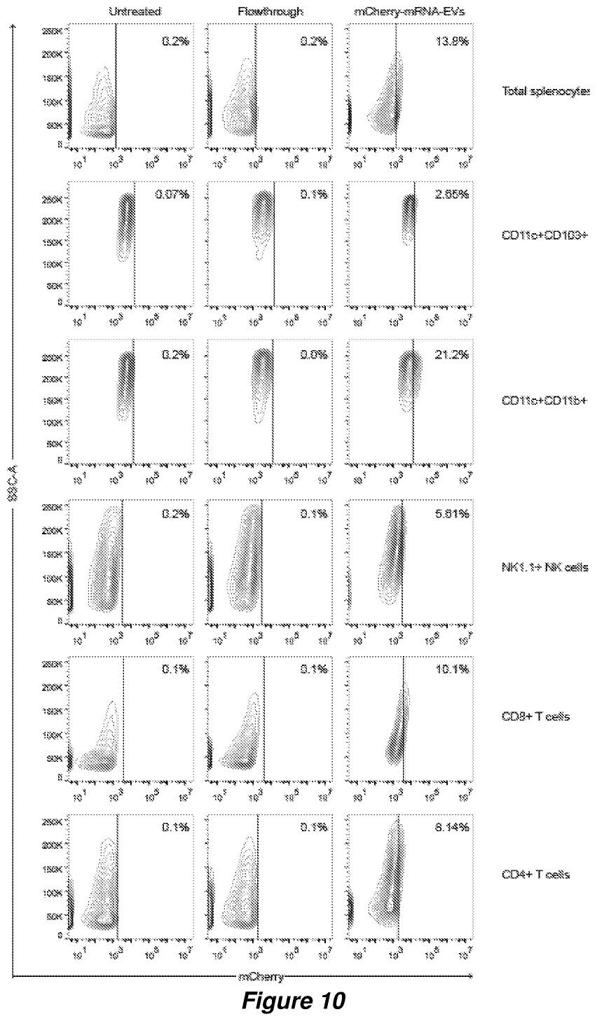

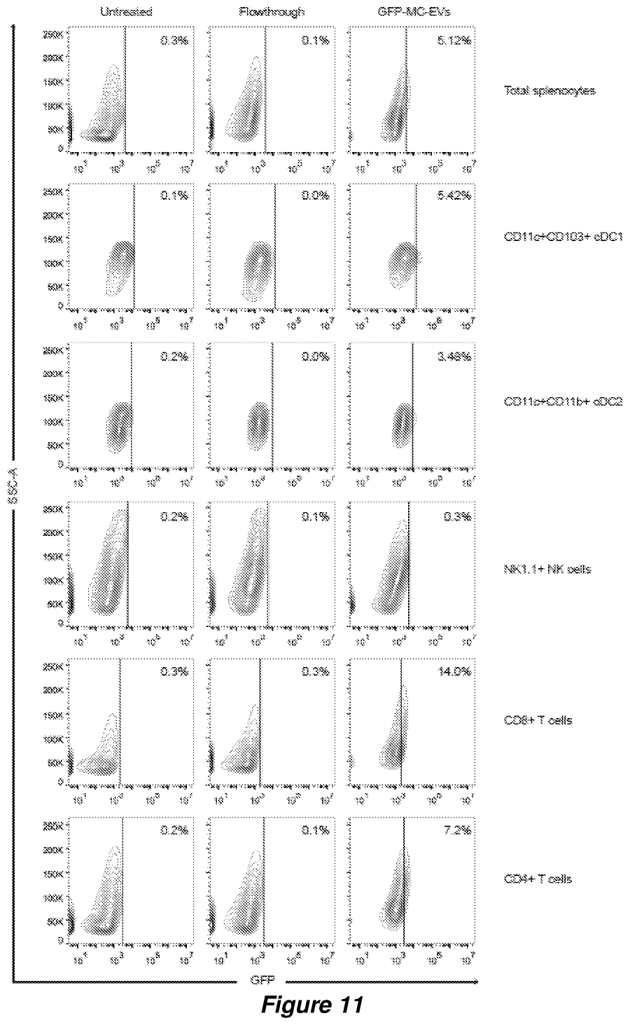

To examine the potential of RBCEVs to deliver nucleic acid payloads to immune cells in the spleen, we incubated splenocytes from immunocompetent C57BL/6 mice with mCherry-mRNA or GFP-MC loaded RBCEVs. After 48 hours, the cells were washed with PBS and incubated with antibodies recognizing several immune cell populations. mCherry expression was found in 13.8% total splenocytes, 2.65% CD11c+ CD103+ DCs, 21.2% CD11c+CD11b+ DCs, 5.6% NK cells, 10% CD8+ T cells and 8% CD4+ T cells (FIG. 10). GFP expression was found in 5% total splenocytes, 5.4% CD11c+CD103+ DCs, 3.5% CD11c+CD11 b+ DCs, 0.3% NK cells, 14% CD8+ T cells and 7.2% CD4+ T cells (FIG. 11). These data suggest that RBCEVs are able to deliver nucleic acid to immune cells in the spleen for transgene expression.

### Example 2

Genome Editing with RNA-Loaded RBCEVs in T Lymphocytes

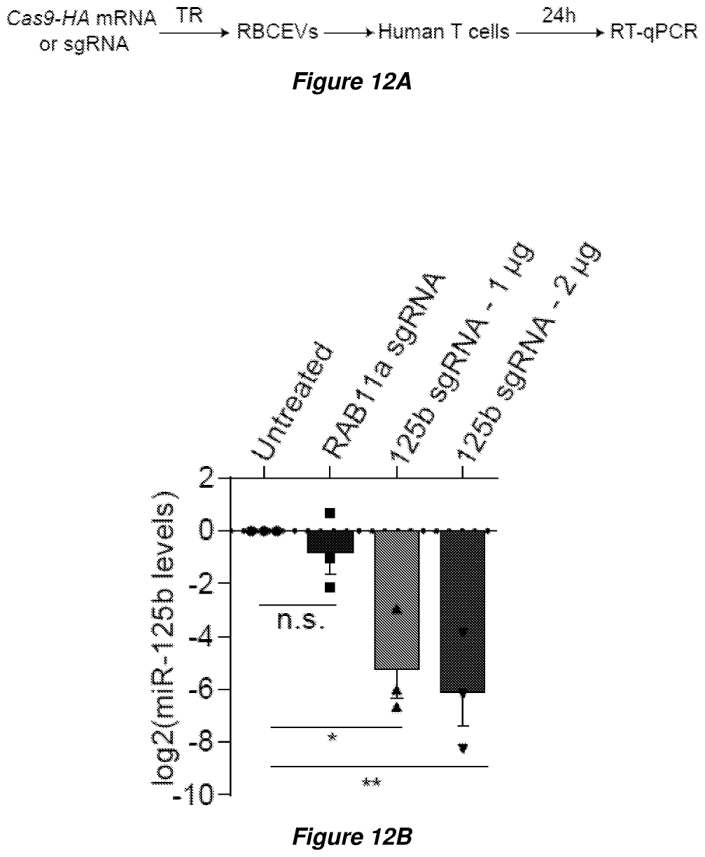

We loaded RBCEVs with Cas9-HA mRNA and a guide RNA (sgRNA) targeting the mir-125b2 locus or a sgRNA targeting the RAB11a locus as a negative control (FIG. 12A). After 24h, cells were collected for RT-qPCR to quantify mature miR-125b levels. Targeting the mir-125b2 locus using Cas9-HA mRNA and 125b sgRNA at different concentrations led to a significant decrease in cellular levels of miR-125b while no effect was observed in cells treated with sgRNA targeting RAB11a, as compared to untreated samples (FIG. 12B). This data implies that we are able to perform genome editing with RNA-loaded RBCEVs.

Delivery of mRNA into Mouse Bone Marrow-Derived Dendritic Cells (BMDCs) Via RBCEVs

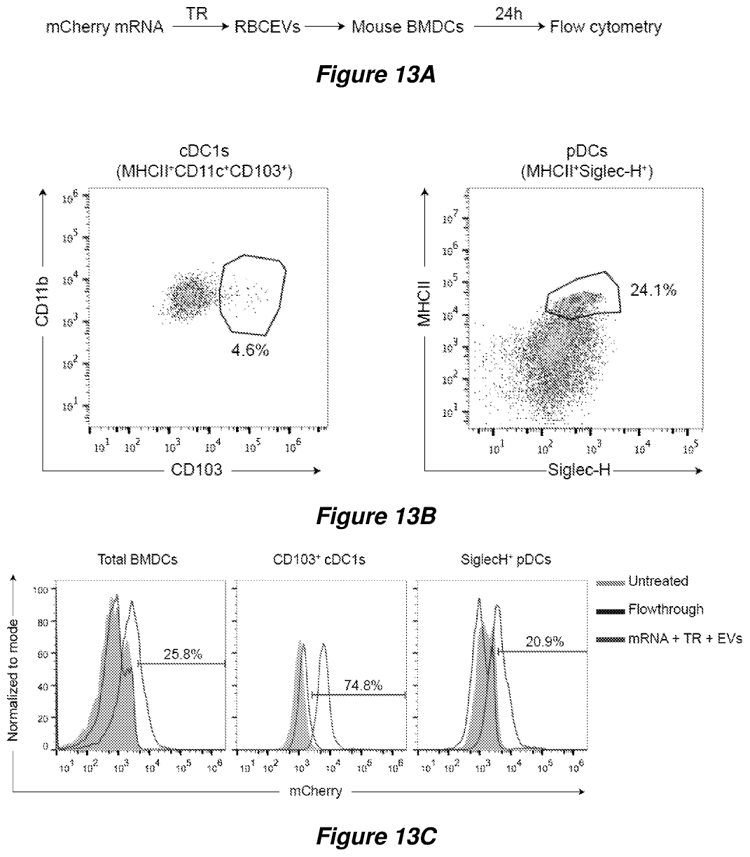

We collected bone marrow cells from the femurs of 6-week-old C57BL/6J mice and cultured them in IMDM supplemented with 2% FBS, 5 ng/mL FLT3L, and 5 ng/mL GM-CSF for 15 days. Flow cytometry analysis confirmed the differentiation of mouse bone marrow cells to dendritic cells (FIG. 13B). About 5% of the cells were conventional dendritic cells type 1 (cDC1s) while about 24% of the cells maturated into plasmacytoid dendritic cells (pDCs) (FIG. 13B). Differentiated BMDCs were incubated with RBCEVs loaded with mCherry mRNA (FIG. 13A). After 24h, we collected BMDCs and analyzed mCherry fluorescence using flow cytometry. About 26% of the total BMDCs were positive for mCherry (FIG. 13C). Conventional dendritic cells type 1 (cDC1s) had the highest proportion of mCherry positive cells (74.8%) while one fifth of plasmacytoid dendritic cells (pDCs) were also positive for mCherry (FIG. 13C). These results indicate that RBCEVs could also serve as a platform for RNA delivery in DCs.

### In Vivo Delivery of Small RNAs to Immune Cells

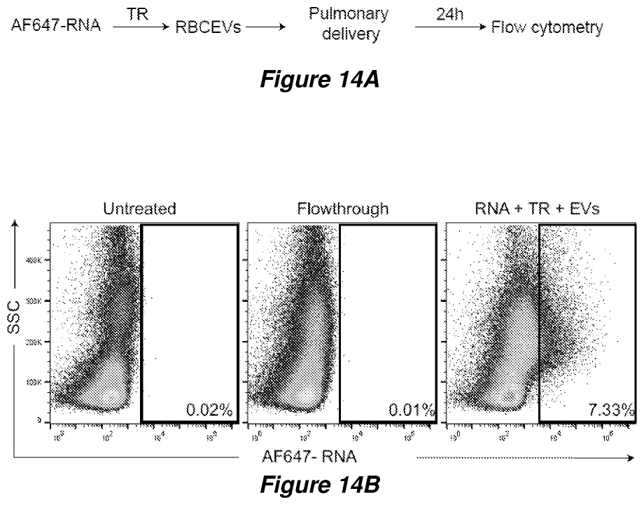

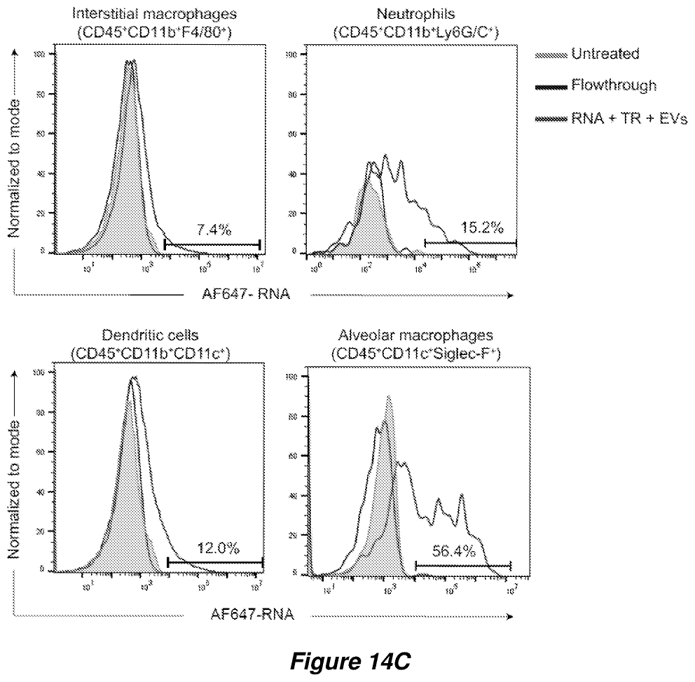

We loaded RBCEVs with a non-targeting siRNA labelled with AF647 fluorescence to track the uptake of the RNA in vivo by pulmonary delivery of the EVs in C57BL/6 mice (FIG. 14A). 24 h after RBCEV administration in the lung, we dissociated lung cells and analyzed different immune cell types using flow cytometry. Overall, about 7.3% of the lung cells were positive for AF647 signal (FIG. 14B). Among the immune cells, about 7.4% of macrophages took up the RNA-loaded EVs. Dendritic cells and neutrophils also engulfed the labelled EVs at comparable percentages (12% and 15.2%, respectively) (FIG. 14C). Alveolar macrophages were the dominant recipients of RNA-loaded EVs with more than half of the total cells (56.4%) were positive for AF647 signal (FIG. 14C). Taken together, these data suggest that we can deliver RNAs to tissue resident immune cells in vivo using RBCEVs.

### REFERENCES

A number of publications are cited above in order to more fully describe and disclose the invention and the state of the art to which the invention pertains. Full citations for these references are provided below. The entirety of each of these references is incorporated herein.
1. Sadelain, M., Rivière, I. & Riddell, S. Therapeutic T cell engineering. Nature 545, 423 (2017).
2. Zhang, Z., Qiu, S., Zhang, X. & Chen, W. Optimized DNA electroporation for primary human T cell engineering. BMC Biotechnol. 18, 4-4 (2018).
3. Roth, T. L. et al. Reprogramming human T cell function and specificity with non-viral genome targeting. Nature 559, 405-409 (2018).
4. van Niel, G., D'Angelo, G. & Raposo, G. Shedding light on the cell biology of extracellular vesicles. Nat. Rev. Mol. Cell Biol. 19, 213 (2018).
5. Meldolesi, J. Exosomes and Ectosomes in Intercellular Communication. Curr. Biol. 28, R435-R444 (2018).
6. Usman, W. M. et al. Efficient RNA drug delivery using red blood cell extracellular vesicles. Nat. Commun. 9, 2359 (2018).
7. Wissink, E. M., Smith, N. L., Spektor, R., Rudd, B. D. & Grimson, A. MicroRNAs and Their Targets Are Differentially Regulated in Adult and Neonatal Mouse CD8+ T Cells. Genetics 201, 1017-1030 (2015).

For standard molecular biology techniques, see Sambrook, J., Russel, D. W. Molecular Cloning, A Laboratory Manual. 3 ed. 2001, Cold Spring Harbor, N.Y.: Cold Spring Harbor Laboratory Press

1. A method for delivering a nucleic acid into an immune cell, the method comprising incubating the immune cell with a red blood cell extracellular vesicle (RBCEV) loaded with a nucleic acid cargo.

2. A method of transducing an immune cell with a nucleic acid, the method comprising incubating the immune cell with a RBCEV loaded with a nucleic acid cargo.

3. The method according to claim 1 or claim 2, wherein the immune cell is a peripheral blood mononuclear cell (PBMC).

4. The method according to any one of claims 1 to 3, wherein the immune cell is a CD3+ cell.

5. The method according to any one of claims 1 to 4, wherein the immune cell is a T cell.

6. The method according to any one of claims 1 to 4, wherein the immune cell is a dendritic cell.

7. The method according to any one of claims 1 to 6, wherein the method is performed in vitro or ex vivo.

8. The method according to any one of claims 1 to 7, wherein the method comprises a step of loading the RBCEV with a nucleic acid cargo.

9. The method according to claim 8, wherein the loading step is performed in vitro or ex vivo.

10. The method according to any one of claims 1 to 9, wherein the nucleic acid cargo comprises RNA.

11. The method according to any one of claims 1 to 10, wherein the nucleic acid cargo is selected from the group consisting of an antisense oligonucleotide, a messenger RNA, a siRNA, a miRNA, or a plasmid.

12. The method according to any one of claims 1 to 11, wherein the method comprises an initial step of isolating an immune cell from a subject.

13. The method according to any one of claims 1 to 12, wherein the method comprises administering to a subject the immune cell comprising the RBCEV loaded with a nucleic acid cargo.

14. Use of a RBCEV loaded with a nucleic acid cargo for delivering the nucleic acid into an immune cell.

15. An immune cell comprising an exogenous nucleic acid, wherein the nucleic acid is or has been delivered using a RBCEV.

16. A composition comprising one or more immune cells comprising an exogenous nucleic acid, wherein the nucleic acid is or has been delivered using a RBCEV.

17. A method of treatment comprising administering to a subject in need of treatment a therapeutically effective amount of an immune cell according to claim 15 or a composition according to claim 16.

18. An immune cell according to claim 15 or a composition according to claim 16 for use in a method of treatment.

19. Use of an immune cell according to claim 15 or a composition according to claim 16 in the manufacture of a medicament for the treatment of a disease or disorder.
